(19) United States
(12) Patent Application Publication
(10) Pub. No.: US 2022/0367059 A1
(43) Pub. Date: Nov. 17, 2022

(54) SYSTEM AND METHOD FOR WELLNESS ASSESSMENT OF A PET

(71) Applicant: MARS, INCORPORATED, McLean, VA (US)

(72) Inventors: Robert MOTT, San Francisco, CA (US); Xin YANG, San Francisco, CA (US); Adam PASSEY, San Francisco, CA (US); Shao En HUANG, San Francisco, CA (US); Nathanael YODER, San Francisco (UA); Robert CHAMBERS, San Francisco, CA (US); Aletha CARSON, Portland, OR (US); Scott LYLE, San Francisco, CA (US); David ALLEN, San Francisco, CA (US); Christian JUNGE, San Francisco, CA (US)

(73) Assignee: MARS, INCORPORATED, McLean, VA (US)

(21) Appl. No.: 17/621,670

(22) PCT Filed: Jun. 26, 2020
(86) PCT No.: PCT/US2020/039909

§ 371 Date: Dec. 21, 2021

Related U.S. Application Data
(60) Provisional application No. 63/007,896, filed on Apr. 9, 2020.
(60) Provisional application No. 62/970,575, filed on Feb. 5, 2020.
(60) Provisional application No. 62/867,226, filed on Jun. 26, 2019.

(51) Int. Cl. G16H 50/30; G16H 50/20

(52) U.S. Cl. CPC G16H 50/30; G16H 50/20

(57) ABSTRACT

A system, method, and apparatus for assessing pet wellness. The method includes receiving data related to a pet. The method also includes determining based on the data one or more health indicators of the pet, and performing a wellness assessment of the pet based on the one or more health indicators. In addition, the method includes determining a recommendation to a pet owner based on the wellness assessment. The method further includes transmitting the recommendation to a mobile device of the pet owner, wherein the recommendation is displayed at the mobile device to the pet owner.

### CROSS-REFERENCE TO RELATED APPLICATIONS

This application claims priority to U.S. Patent Application Ser. No. 62/866,225, filed on Jun. 26, 2019, U.S. Patent Application Ser. No. 62/970,575, filed on Feb. 5, 2020, and U.S. Patent Application Ser. No. 63/007,896, filed Apr. 9, 2020, which are incorporated herein by reference in their entirety.

### FIELD

The embodiments described in the disclosure relate to data analysis. For example, some non-limiting embodiments relate to data analysis of pet activity or other data.

### BACKGROUND

Mobile devices and/or wearable devices have been fitted with various hardware and software components that can help track human location. For example, mobile devices can communicate with a global positioning system (GPS) to help determine their location. More recently, mobile devices and/or wearable devices have moved beyond mere location tracking and can now include sensors that help to monitor human activity. The data resulting from the tracked location and/or monitored activity can be collected, analyzed and displayed. For example, a mobile device and/or wearable devices can be used to track the number of steps taken by a human for a preset period of time. The number of steps can then be displayed on a user graphic interface of the mobile device or wearable device.

The ever-growing emphasis on pet safety and health has resulted in an increased need to monitor pet behavior. Accordingly, there is an ongoing demand in the pet product industry for a system and/or method for tracking and monitoring pet activity. In particular, there remains a need for a wearable pet device that can accurately track the location of a pet, while also monitoring pet activity.

### BRIEF SUMMARY

To remedy the aforementioned deficiencies, the disclosure presents systems, methods, and apparatuses which can be used to analyze data. For example, certain non-limiting embodiments can be used to monitor and track pet activity.

In certain non-limiting embodiments, the disclosure describes a method for monitoring pet activity. The method includes monitoring a location of a wearable device. The method also includes determining that the wearable device has exited a geo-fence zone based on the location of the wearable device. In addition, the method includes instructing the wearable device to turn on an indicator after determining that the wearable device has exited the geo-fence zone. The indicator can be at least one of an illumination device, a sound device, or a vibrating device. Further, the method can include determining that the wearable device has entered the geo-fence zone and turning off the indicator when the wearable device has entered the geo-fence zone.

In certain non-limiting embodiments, the disclosure describes a method for monitoring pet activity. The method includes receiving data related to a pet from a wearable device comprising a sensor. The method also includes determining, based on the data, one or more health indicators of the pet and performing a wellness assessment of the pet based on the one or more health indicators of the pet. In addition, the method can include transmitting the wellness assessment of the pet to a mobile device. The wellness assessment of the pet can be displayed at the mobile device to a user. The method can be performed by the wearable device, one or more servers, a cloud-computing platform and/or any combination thereof.

In some non-limiting embodiments, the disclosure describes a method that can include receiving data at an apparatus. The method can also include analyzing the data using two or more layer modules, wherein each of the layer modules includes at least one of a many-to-many approach, striding, downsampling, pooling, multi-scaling, or batch normalization. In addition, the method can include determining an output based on the analyzed data. The data can include at least one of financial data, cyber security data, electronic health records, health data, image data, video data, acoustic data, human activity data, pet activity data and/or any combination thereof. The output can include one or more of the following: a wellness assessment, a health recommendation, a financial prediction, a security recommendation, image or video recognition, sound recognition and/or any combination thereof. The determined output can be displayed on a mobile device.

In certain non-limiting embodiments, the disclosure describes a method for assessing pet wellness. The method can include receiving data related to a pet and determining, based on the data, one or more health indicators of the pet. The method can also include performing a wellness assessment of the pet based on the one or more health indicators. In addition, the method can include providing or determining a recommendation to a pet owner based on the wellness assessment. The method can further include transmitting the recommendation to a mobile device of the pet owner, wherein the recommendation is displayed at the mobile device of the pet owner.

In certain non-limiting embodiments, an apparatus for monitoring pet activity can include at least one memory comprising computer program code and at least one processor. The computer program code can be configured, when executed by the at least one processor, to cause the apparatus to receive data related to a pet from a wearable device comprising a sensor. The computer program code can also be configured, when executed by the at least one processor, to cause the apparatus to determine, based on the data, one or more health indicators of the pet, and to perform a wellness assessment of the pet based on the one or more health indicators of the pet. In addition, the computer program code can also be configured, when executed by the at least one processor, to cause the apparatus to transmit the wellness assessment of the pet to a mobile device. The wellness assessment of the pet is displayed at the mobile device to a user.

Certain non-limiting embodiments can be directed to a wearable device. The wearable device can include a housing that includes a top cover. The housing can also comprise a base couple with the top cover. The housing can include a sensor for monitoring data related to a pet. The housing can also include a transceiver for transmitting the data related to the pet. Further, the housing can include an indicator, where the indicator is at least one of an illumination device, a sound device, a vibrating device and/or any combination thereof.

According to certain non-limiting embodiments, at least one non-transitory computer-readable medium encoding instruction is provided that, when executed in hardware, performs a process according to the methods disclosed herein. In some non-limiting embodiments, an apparatus can include a computer program product encoding instructions for processing data of a tested pet product according to the above method. In other embodiments, a computer program product can encode instructions for performing a process according to the methods disclosed herein.

It is to be understood that both the foregoing general description and the following detailed description are exemplary and are intended to provide further explanation of the disclosed subject matter claimed.

### BRIEF DESCRIPTION OF THE DRAWINGS

The foregoing and other objects, features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 illustrates a system used to track and monitor a pet according to certain non-limiting embodiments;

FIG. 2 illustrates a device that can be used to track and monitor a pet according to certain non-limiting embodiments;

FIG. 3 is a logical block diagram illustrating a device that can be used to track and monitor a pet according to certain non-limiting embodiments;

FIG. 4 is a flow diagram illustrating a method for tracking a pet according to certain non-limiting embodiments;

FIG. 5 is a flow diagram illustrating a method for tracking and monitoring the pet according to certain non-limiting embodiments.

FIG. 6 illustrates an example of two deep learning models according to certain non-limiting embodiments.

FIGS. 7(a), 7(b), and 7(c) illustrate a model architecture according to certain non-limiting embodiments.

FIG. 8 illustrates examples of a model according to certain non-limiting embodiments.

FIG. 9 illustrates an example embodiment of the models shown in FIG. 8.

FIG. 10 illustrates an example architecture of one or more of the models shown in FIG. 8.

FIG. 11 illustrates an example of model parameters according to certain non-limiting embodiments.

FIG. 12 illustrates an example of a representative model train run according to certain non-limiting embodiments.

FIG. 13 illustrates performance of example models according to certain non-limiting embodiments.

FIG. 14 illustrates a heatmap showing performance of a model according to certain non-limiting embodiments.

FIG. 15 illustrates performance metrics of a model according to certain non-limiting embodiments.

FIG. 16 illustrates performance of an n-fold ensembled ms-C/L model according to certain non-limiting embodiments.

FIG. 17 illustrates the effects of changing the sliding window length used in the interference step according to certain non-limiting embodiments.

FIG. 18 illustrates performance of one or more models according to certain non-limiting embodiments based on a number of sensors.

FIG. 19 illustrates performance analysis of models according to certain non-limiting embodiments.

FIG. 20 illustrates a flow diagram of a process for assessing pet wellness according to certain non-limiting embodiments.

FIG. 21 illustrates an example step performed during the process for assessing pet wellness according to certain non-limiting embodiments.

FIG. 22 illustrates an example step performed during the process for assessing pet wellness according to certain non-limiting embodiments.

FIG. 23 illustrates an example step performed during the process for assessing pet wellness according to certain non-limiting embodiments.

FIG. 24 illustrates an example step performed during the process for assessing pet wellness according to certain non-limiting embodiments.

FIG. 25A illustrates a flow diagram of a process for assessing pet wellness according to certain non-limiting embodiments.

FIG. 25B illustrates a flow diagram of a process for assessing pet wellness according to certain non-limiting embodiments.

FIG. 26 illustrates a flow diagram of a process for assessing pet wellness according to certain non-limiting embodiments

FIG. 27 illustrates a perspective view of a collar having a tracking device and a band, according to an embodiment the disclosed subject matter.

FIG. 28 illustrates a perspective view of the tracking device of FIG. 27, according to the disclosed subject matter.

FIG. 29 illustrates a front view of the tracking device of FIG. 27, according to the disclosed subject matter.

FIG. 30 illustrates an exploded view of the tracking device of FIG. 27.

FIG. 31 depicts a left side view of the tracking device of FIG. 27, with the right side being identical to the left side view.

FIG. 32 depicts a top view of the tracking device of FIG. 27, with the bottom view being identical to the top side view.

FIG. 33 depicts a back view of the tracking device of FIG. 27.

FIG. 34 illustrates a perspective view of a tracking device according to another embodiment of the disclosed subject matter.

FIG. 35 illustrates a front view of the tracking device of FIG. 34, according to the disclosed subject matter.

FIG. 36 illustrates an exploded view of the tracking device of FIG. 34.

FIG. 37 illustrates a front view of the tracking device of FIG. 34, according to the disclosed subject matter.

FIG. 38 depicts a left side view of the tracking device of FIG. 34, with the right side being identical to the left side view.

FIG. 39 depicts a top view of the tracking device of FIG. 34, with the bottom view being identical to the top side view.

FIG. 40 depicts a back view of the tracking device of FIG. 34.

FIG. 41 depicts a back view of the tracking device couplable with a cable, according to the disclosed subject matter.

FIG. 42 depicts a collar having a receiving plate to receiving a tracking device, according to the disclosed subject matter.

FIGS. 43 and 44 depict a pet wearing a collar, according to embodiments of the disclosed subject matter.

FIG. 45 depicts a collar receiving plate and/or support frame to receive a tracking device, according to another aspect of the disclosed subject matter.

FIG. 46 depicts a collar receiving plate and/or support frame to receive a tracking device, according to another aspect of the disclosed subject matter.

FIG. 47 depicts a collar receiving plate and/or support frame to receive a tracking device, according to another aspect of the disclosed subject matter.

### DETAILED DESCRIPTION

There remains a need for a system, method, and device that can monitor and track pet activity. The presently disclosed subject matter addresses this need, as well as others needs associated with the health and wellness of pets. Specifically, data related to the tracked or monitored activity of a pet can be collected and used to detect any potential health risks related to the pet. The identified potential health risks, as well as a summary of the collected data, can then be transmitted to and/or displayed for or by a pet owner.

U.S. patent application Ser. No. 15/291,882, now U.S. Pat. No. 10,142,773 B2, U.S. patent application Ser. No. 15/287,544, U.S. patent application Ser. No. 14/231,615, U.S. Provisional Application Nos. 62/867,226, 62/768,414, and 62/970,575, U.S. Design application Nos. 29/696,311 and 29/696,315 are hereby incorporated by reference. The entire subject matter disclosed in the above referenced applications, including the specification, claims, and figures are incorporated herein.

The present disclosure will now be described more fully hereinafter with reference to the accompanying drawings, which form a part hereof, and which show, by way of illustration, certain example embodiments. Subject matter can, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter can be embodied as methods, devices, components, or systems. Accordingly, embodiments can, for example, take the form of hardware, software, firmware or any combination thereof (other than software per se). The following detailed description is, therefore, not intended to be taken in a limiting sense.

In the detailed description herein, references to “embodiment,” “an embodiment,” “one non-limiting embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In general, terminology can be understood at least in part from usage in context. For example, terms, such as “and”, “or”, or “and/or,” as used herein can include a variety of meanings that can depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, can be used to describe any feature, structure, or characteristic in a singular sense or can be used to describe combinations of features, structures or characteristics in a plural sense. Similarly, terms, such as “a,” “an,” or “the,” again, can be understood to convey a singular usage or to convey a plural usage, depending at least in part upon context. In addition, the term “based on” can be understood as not necessarily intended to convey an exclusive set of factors and can, instead, allow for existence of additional factors not necessarily expressly described, again, depending at least in part on context.

As used herein, the terms “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

These computer program instructions can be provided to a processor of: a general purpose computer to alter its function to a special purpose; a special purpose computer; ASIC; or other programmable digital data processing apparatus, such that the instructions, which execute via the processor of the computer or other programmable data processing apparatus, implement the functions/acts specified in the block diagrams or operational block or blocks, thereby transforming their functionality in accordance with embodiments herein.

For the purposes of this disclosure a computer readable medium (or computer-readable storage medium/media) stores computer data, which data can include computer program code (or computer-executable instructions) that is executable by a computer, in machine readable form. By way of example, and not limitation, a computer readable medium can comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors, such as an elastic computer cluster, and associated network and storage devices, as well as operating software and one or more database systems and application software that support the services provided by the server. The server, for example, can be a cloud-based server, a cloud-computing platform, or a virtual machine. Servers can vary widely in configuration or capabilities, but generally a server can include one or more central processing units and memory. A server can also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

For the purposes of this disclosure a “network” should be understood to refer to a network that can couple devices so that communications can be exchanged, such as between a server and a client device or other types of devices, including between wireless devices coupled via a wireless network, for example. A network can also include mass storage, such as network attached storage (NAS), a storage area network (SAN), or other forms of computer or machine-readable media, for example. A network can include the Internet, one or more local area networks (LANs), one or more wide area networks (WANs), wire-line type connections, wireless type connections, cellular or any combination thereof. Likewise, sub-networks, which can employ differing architectures or can be compliant or compatible with differing protocols, can interoperate within a larger network. Various types of devices can, for example, be made available to provide an interoperable capability for differing architectures or protocols. As one illustrative example, a router can provide a link between otherwise separate and independent LANs.

A communication link or channel can include, for example, analog telephone lines, such as a twisted wire pair, a coaxial cable, full or fractional digital lines including T1, T2, T3, or T4 type lines, Integrated Services Digital Networks (ISDNs), Digital Subscriber Lines (DSLs), wireless links including satellite links, or other communication links or channels, such as can be known to those skilled in the art. Furthermore, a computing device or other related electronic devices can be remotely coupled to a network, such as via a wired or wireless line or link, for example.

For purposes of this disclosure, a “wireless network” should be understood to couple client devices with a network. A wireless network can employ stand-alone ad-hoc networks, mesh networks, wireless land area network (WLAN), cellular networks, or the like. A wireless network can further include a system of terminals, gateways, routers, or the like coupled by wireless radio links, or the like, which can move freely, randomly or organize themselves arbitrarily, such that network topology can change, at times even rapidly.

A wireless network can further employ a plurality of network access technologies, including Wi-Fi, Long Term Evolution (LTE), WLAN, Wireless Router (WR) mesh, or 2nd, 3rd, 4^{th}, 5th generation (2G, 3G, 4G, or 5G) cellular technology, or the like. Network access technologies can allow wide area coverage for devices, such as client devices with varying degrees of mobility, for example.

For example, a network can allow RF or wireless type communication via one or more network access technologies, such as Global System for Mobile communication (GSM), Universal Mobile Telecommunications System (UMTS), General Packet Radio Services (GPRS), Enhanced Data GSM Environment (EDGE), 3GPP LTE, LTE Advanced, Wideband Code Division Multiple Access (WCDMA), Bluetooth, 802.11b/g/n, or the like. A wireless network can include virtually any type of wireless communication mechanism by which signals can be communicated between devices, such as a client device or a computing device, between or within a network, or the like.

A computing device can be capable of sending or receiving signals, such as via a wired or wireless network, or can be capable of processing or storing signals, such as in memory as physical memory states, and can, therefore, operate as a server. Thus, devices capable of operating as a server can include, as examples, dedicated rack-mounted servers, desktop computers, laptop computers, set top boxes, integrated devices combining various features, such as two or more features of the foregoing devices, or the like. Servers can vary widely in configuration or capabilities, but generally a server can include one or more central processing units and memory. A server can also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

In certain non-limiting embodiments, a wearable device can include one or more sensors. The term “sensor” can refer to any hardware or software used to detect a variation of a physical quantity caused by activity or movement of the pet, such as an actuator, a gyroscope, a magnetometer, microphone, pressure sensor, or any other device that can be used to detect an object's displacement. In one non-limiting example, the sensor can be a three-axis accelerometer. The one or more sensors or actuators can be included in a microelectromechanical system (MEMS). A MEMS, also referred to as a MEMS device, can include one or more miniaturized mechanical and/or electro-mechanical elements that function as sensors and/or actuators and can help to detect positional variations, movement, and/or acceleration. In other embodiments any other sensor or actuator can be used to detect any physical characteristic, variation, or quantity. The wearable device, also referred to as a collar device, can also include one or more transducers. The transducer can be used to transform the physical characteristic, variation, or quantity detected by the sensor and/or actuator into an electrical signal, which can be transmitted from the one or more wearable device through a network to a server.

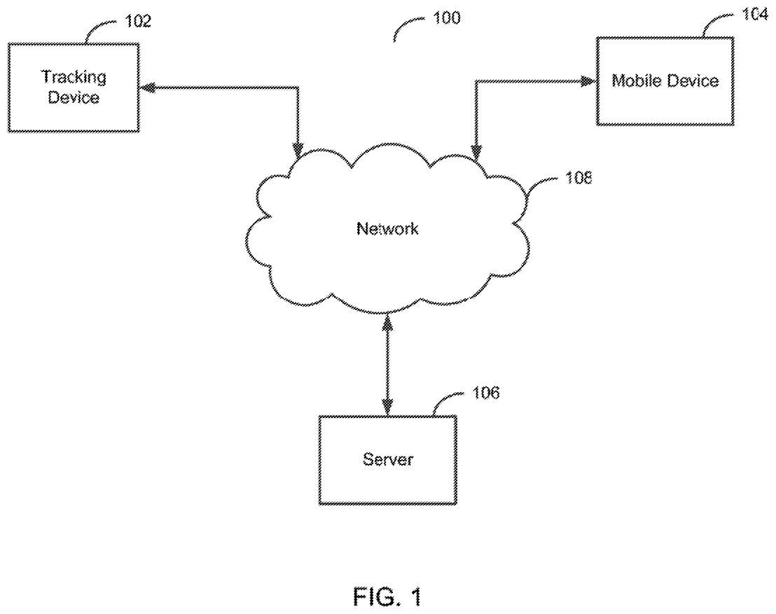

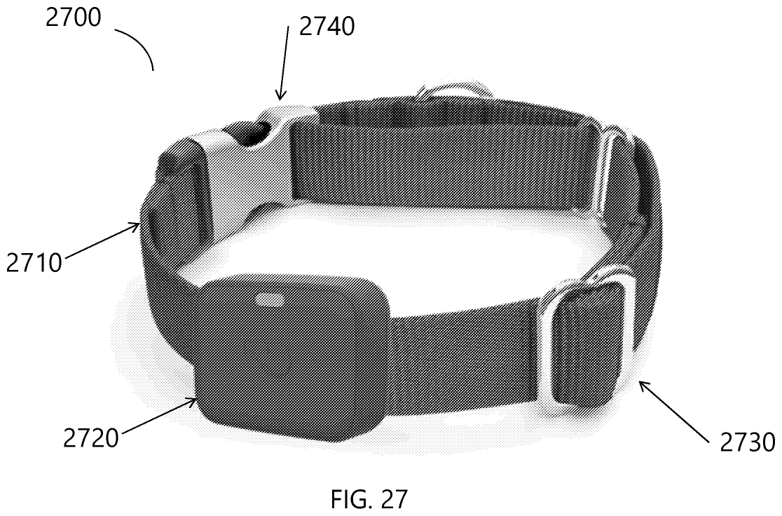

FIG. 1 illustrates a system diagram used to track and monitor a pet according to certain non-limiting embodiments. In particular, as illustrated in FIG. 1, the system 100 can include a tracking device 102, a mobile device 104, a server 106, and/or a network 108. Tracking device 102 can be a wearable device as shown in FIGS. 27-47. The wearable device can be placed on a collar of the pet, and can be used to track, monitor, and/or detect the activity of the pet using one or more sensors.

As illustrated in FIG. 1, a tracking device 102 can comprise a computing device designed to be worn, or otherwise carried, by a user or other entity, such as a pet or animal. The terms “animal” or “pet” as used in accordance with the present disclosure can refer to domestic animals including, domestic dogs, domestic cats, horses, cows, ferrets, rabbits, pigs, rats, mice, gerbils, hamsters, goats, and the like. Domestic dogs and cats are particular non-limiting examples of pets. The term “animal” or “pet” as used in accordance with the present disclosure can also refer to wild animals, including, but not limited to bison, elk, deer, venison, duck, fowl, fish, and the like.

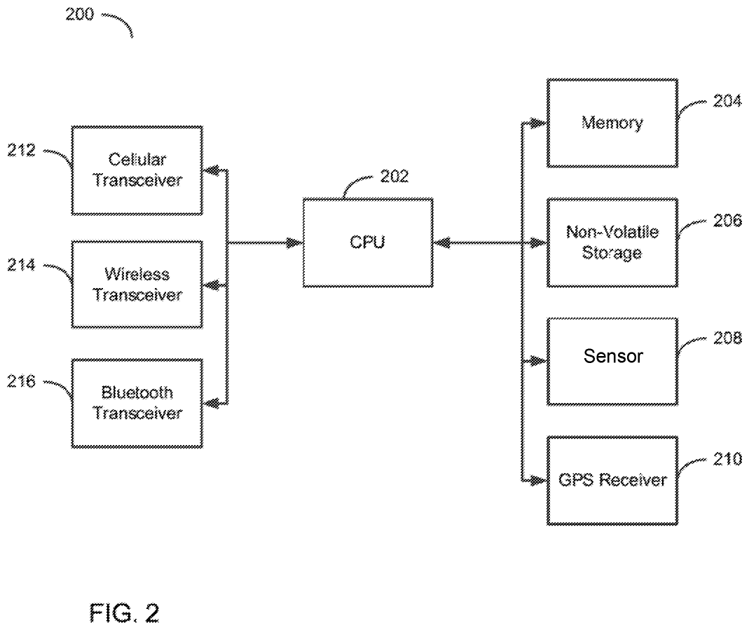

In one non-limiting embodiment, tracking device 102 can include the hardware illustrated in FIG. 2. The tracking device 102 can be configured to collect data generated by various hardware or software components, generally referred to as sensors, present within the tracking device 102. For example, a GPS receiver or one or more sensors, such as accelerometer, gyroscope, or any other device or component used to record, collect, or receive data regarding the movement or activity of the tracking device 102. The activity of tracking device 102, in some non-limiting embodiments, can mimic the movement of the pet on which the tracking device is located. While tracking device 102 can be attached to the collar of the pet, as described in U.S. patent application Ser. No. 14/231,615, hereby incorporated by reference in its entirety, in other embodiments tracking device 102 can be attached to any other item worn by the pet. In some non-limiting embodiments, tracking device 102 can be located on or inside the pet itself, such as, for example, a microchip implanted within the pet.

As discussed in more detail herein, tracking device 102 can further include a processor capable of processing the one or more data collected from tracking device 102. The processor can be embodied by any computational or data processing device, such as a central processing unit (CPU), digital signal processor (DSP), application specific integrated circuit (ASIC), programmable logic devices (PLDs), field programmable gate arrays (FPGAs), digitally enhanced circuits, or comparable device or a combination thereof. The processors can be implemented as a single controller, or a plurality of controllers or processors. In some non-limiting embodiments, the tracking device 102 can specifically be configured to collect, sense, or receive data, and/or pre-process data prior to transmittal. In addition to sensing, recording, and/or processing data, tracking device 102 can further be configured to transmit data, including location and any other data monitored or tracked, to other devices or severs via network 108. In certain non-limiting embodiments, tracking device 102 can transmit any data tracked or monitored data continuously to the network. In other non-limiting embodiments, tracking device 102 can discretely transmit any tracked or monitored data. Discrete transmittal can be transmitting data after a finite period of time. For example, tracking device 102 can transmit data once an hour. This can help to reduce the battery power consumed by tracking device 102, while also conserving network resources, such as bandwidth.

As shown in FIG. 1, tracking device 102 can communicate with network 108. Although illustrated as a single network, network 108 can comprise multiple or a plurality of networks facilitating communication between devices. Network 108 can be a radio-based communication network that uses any available radio access technology. Available radio access technologies can include, for example, Bluetooth, wireless local area network (“WLAN”), Global System for Mobile Communications (GMS), Universal Mobile Telecommunications System (UMTS), any Third Generation Partnership Project (“3GPP”) Technology, including Long Term Evolution (“LTE”), LTE-Advanced, Third Generation technology (“3G”), or Fifth Generation (“5G”)/New Radio (“NR”) technology. Network 108 can use any of the above radio access technologies, or any other available radio access technology, to communicate with tracking device 102, server 106, and/or mobile device 104.

In one non-limiting embodiment, the network 108 can include a WLAN, such as a wireless fidelity (“Wi-Fi”) network defined by the IEEE 802.11 standards or equivalent standards. In this embodiment, network 108 can allow the transfer of location and/or any tracked or monitored data from tracking device 102 to server 106. Additionally, the network 108 can facilitate the transfer of data between tracking device 102 and mobile device 104. In an alternative embodiment, the network 108 can comprise a mobile network such as a cellular network. In this embodiment, data can be transferred between the illustrated devices in a manner similar to the embodiment wherein the network 108 is a WLAN. In certain non-limiting embodiments tracking device 102, also referred to as wearable device, can reduce network bandwidth and extend battery life by transmitting when data to server 106 only or mostly when it is connected to the WLAN network. When it is not connected to a WLAN, tracking device 102 can enter a power-save mode where it can still monitor and/or track data, but not transmit any of the collected data to server 106. This can also help to extend the battery life of tracking device 102.

In one non-limiting embodiment, tracking device 102 and mobile device 104 can transfer data directly between the devices. Such direct transfer can be referred to as device-to-device communication or mobile-to-mobile communication. While described in isolation, network 108 can include multiple networks. For example, network 108 can include a Bluetooth network that can help to facilitate transfers of data between tracking device 102 and mobile device 104, a wireless land area network, and a mobile network.

The system 100 can further include a mobile device 104. Mobile device 104 can be any available user equipment or mobile station, such as a mobile phone, a smart phone or multimedia device, or a tablet device. In alternative embodiments, mobile device 104 can be a computer, such as a laptop computer, provided with wireless communication capabilities, personal data or digital assistant (PDA) provided with wireless communication capabilities, portable media player, digital camera, pocket video camera, navigation unit provided with wireless communication capabilities or any combinations thereof. As discussed previously, mobile device 104 can communicate with a tracking device 102. In these embodiments, mobile device 104 can receive location, data related to a pet, wellness assessment, and/or health recommendation from a tracking device 102, server 106, and/or network 108. Additionally, tracking device 102 can receive data from mobile device 104, server 106, and/or network 108. In one non-limiting embodiment, tracking device 102 can receive data regarding the proximity of mobile device 104 to tracking device 102 or an identification of a user associated with mobile device 104. A user associated with mobile device 104, for example, can be an owner of the pet.

Mobile device 104 (or non-mobile device) can additionally communicate with server 106 to receive data from server 106. For example, server 106 can include one or more application servers providing a networked application or application programming interface (API). In one non-limiting embodiment, mobile device 104 can be equipped with one or more mobile or web-based applications that communicates with server 106 via an API to retrieve and present data within the application. In one non-limiting embodiment, server 106 can provide visualizations or displays of location or data received from tracking device 102. For example, visualization data can include graphs, charts, or other representations of data received from tracking device 102.

FIG. 2 illustrates a device that can be used to track and monitor a pet according to certain non-limiting embodiments. The device 200 can be, for example, tracking device 102, server 106, or mobile device 104. Device 200 includes a CPU 202, memory 204, non-volatile storage 206, sensor 208, GPS receiver 210, cellular transceiver 212, Bluetooth transceiver 216, and wireless transceiver 214. The device can include any other hardware, software, processor, memory, transceiver, and/or graphical user interface.

As discussed with respect to FIG. 2, the device 200 can a wearable device designed to be worn, or otherwise carried, by a pet. The device 200 includes one or more sensors 208, such as a three axis accelerometer. The one or more sensors can be used in combination with GPS receiver 210, for example. GPS receiver 210 can be used along with sensor 208 which monitor the device 200 to identify its position (via GPS receiver 210) and its acceleration, for example, (via sensor 208). Although illustrated as single components, sensor 208 and GPS receiver 210 can alternatively each include multiple components providing similar functionality. In certain non-limiting embodiment, GPS receiver 210 can instead be a Global Navigation Satellite System (GLONASS) receiver.

Sensor 208 and GPS receiver 210 generate data as described in more detail herein and transmits the data to other components via CPU 202. Alternatively, or in conjunction with the foregoing, sensor 208 and GPS receiver 210 can transmit data to memory 204 for short-term storage. In one non-limiting embodiment, memory 204 can comprise a random access memory device or similar volatile storage device. Memory 204 can be, for example, any suitable storage device, such as a non-transitory computer-readable medium. A hard disk drive (HDD), random access memory (RAM), flash memory, or other suitable memory.

Alternatively, or in conjunction with the foregoing, sensor 208 and GPS receiver 210 can transmit data directly to non-volatile storage 206. In this embodiment, CPU 202 can access the data (e.g., location and/or event data) from memory 204. In some non-limiting embodiments, non-volatile storage 206 can comprise a solid-state storage device (e.g., a “flash” storage device) or a traditional storage device (e.g., a hard disk). Specifically, GPS receiver 210 can transmit location data (e.g., latitude, longitude, etc.) to CPU 202, memory 204, or non-volatile storage 206 in similar manners. In some non-limiting embodiments, CPU 202 can comprise a field programmable gate array or customized application-specific integrated circuit.

As illustrated in FIG. 2, the device 200 includes multiple network interfaces including cellular transceiver 212, wireless transceiver 214, and Bluetooth transceiver 216. Cellular transceiver 212 allows the device 200 to transmit the data, processed by CPU 202, to a server via any radio access network. Additionally, CPU 202 can determine the format and contents of data transferred using cellular transceiver 212, wireless transceiver 214, and Bluetooth transceiver 216 based upon detected network conditions. Transceivers 212, 214, 216 can each, independently, be a transmitter, a receiver, or both a transmitter and a receiver, or a unit or device that can be configured both for transmission and reception. The transmitter and/or receiver (as far as radio parts are concerned) can also be implemented as a remote radio head which is not located in the device itself, but in a mast, for example.

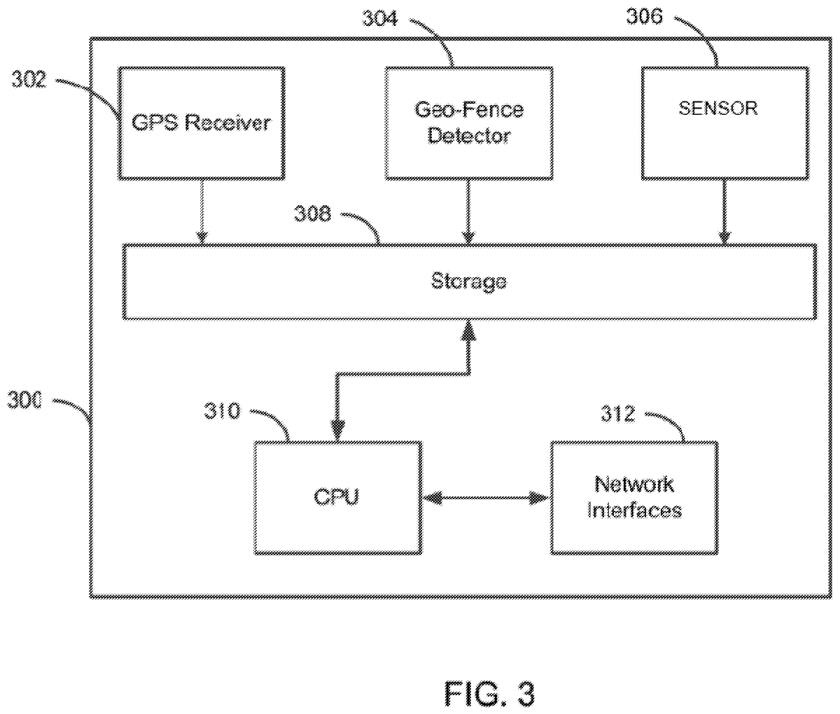

FIG. 3 is a logical block diagram illustrating a device that can be used to track and monitor a pet according to certain non-limiting embodiments. As illustrated in FIG. 3, a device 300, such as tracking device 102 shown in FIG. 1, also referred to as a wearable device, or mobile device 104 shown in FIG. 1, which can include a GPS receiver 302, a geo-fence detector 304, a sensor 306, storage 308, CPU 310, and network interfaces 312. Geo-fence can refer a geolocation-fence as described below. GPS receiver 302, sensor 306, storage 308, and CPU 310 can be similar to GPS receiver 210, sensor 208, memory 204/non-volatile storage 206, or CPU 202, respectively. Network interfaces 312 can correspond to one or more of transceivers 212, 214, 216. Device 300 can also include one or more power sources, such as a battery. Device 300 can also include a charging port, which can be used to charge the battery. The charging port can be, for example, a type-A universal serial bus (“USB”) port, a type-B USB port, a mini-USB port, a micro-USB port, or any other type of port. In some other non-limiting embodiments, the battery of device 300 can be wirelessly charged.

In the illustrated embodiment, GPS receiver 302 records location data associated with the device 300 including numerous data points representing the location of the device 300 as a function of time.

In one non-limiting embodiment, geo-fence detector 304 stores details regarding known geo-fence zones. For example, geo-fence detector 304 can store a plurality of latitude and longitude points for a plurality of polygonal geo-fences. The latitude and/or longitude points or coordinates can be manually inputted by the user and/or automatically detected by the wearable device. In alternative embodiments, geo-fence detector 304 can store the names of known WLAN network service set identifier (SSIDs) and associate each of the SSIDs with a geo-fence, as discussed in more detail with respect to FIG. 4. In non-limiting one non-limiting embodiment, geo-fence detector 304 can store, in addition to an SSID, one or more thresholds for determining when the device 300 exits a geo-fence zone. Although illustrated as a separate component, in some non-limiting embodiments, geo-fence detector 304 can be implemented within CPU 310, for example, as a software module.

In one non-limiting embodiment, GPS receiver 302 can transmit latitude and longitude data to geo-fence detector 304 via storage 308 or, alternatively, indirectly to storage 308 via CPU 310. A geo-fence can be a virtual fence or safe space defined for a given pet. The geo-fence can be defined based on a latitude and/or longitudinal coordinates and/or by the boundaries of a given WLAN connection signal. For example, geo-fence detector 304 receives the latitude and longitude data representing the current location of the device 300 and determines whether the device 300 is within or has exited a geo-fence zone. If geo-fence detector 304 determines that the device 300 has exited a geo-fence zone the geo-fence detector 304 can transmit the notification to CPU 310 for further processing. After the notification has been processed by CPU 310, the notification can be transmitted to the mobile device either directly or via the server.

Alternatively, geo-fence detector 304 can query network interfaces 312 to determine whether the device is connected to a WLAN network. In this embodiment, geo-fence detector 304 can compare the current WLAN SSID (or lack thereof) to a list of known SSIDs. The list of known SSIDs can be based on those WLAN connections that have been previously approved by the user. The user, for example, can be asked to approve an SSID during the set up process for a given wearable device. In another example, the list of known SSIDs can be automatically populated based on those WLAN connections already known to the mobile device of the user. If geo-fence detector 304 does not detect that the device 300 is currently connected to a known SSID, geo-fence detector 304 can transmit a notification to CPU 310 that the device has exited a geo-fence zone. Alternatively, geo-fence detector 304 can receive the strength of a WLAN network and determine whether the current strength of a WLAN connection is within a predetermined threshold. If the WLAN connection is outside the predetermined threshold, the wearable device can be nearing the outer border of the geo-fence. Receiving a notification once a network strength threshold is surpassed can allow a user to receiver a preemptive warning that the pet is about to exit the geo-fence.

As illustrated in FIG. 3, device 300 further includes storage 308. In one non-limiting embodiment, storage 308 can store past or previous data sensed or received by device 300. For example, storage 308 can store past location data. In other non-limiting embodiments, instead of storing previously sensed and/or received data, device 300 can transmit the data to a server, such as server 106 shown in FIG. 1. The previous data can then be used to determine a health indicator which can be stored at the server. The server can then compare the health indicators it has determined based on the recent data it receives to the stored health indicators, which can be based on previously stored data. Alternatively, in certain non-limiting embodiments device 308 can use its own computer capabilities or hardware to determine a health indicator. Tracking changes of the health indicator or metric using device 308 can help to limit or avoid the transmission of data to the server. The wellness assessment and/or health recommendation made by server 106 can be based on the previously stored data. The wellness assessment, for example, can include dermatological diagnoses, such as a flare up, ear infection, arthritis diagnoses, cardiac episode, and/or pancreatic episode.

In one non-limiting example, the stored data can include data describing a walk environment details, which can include the time of day, the location of the tracking device, movement data associated with the device (e.g., velocity, acceleration, etc.) for previous time the tracking device exited a geo-fence zone. The time of day can be determined via a timestamp received from the GPS receiver or via an internal timer of the tracking device.

CPU 310 is capable of controlling access to storage 308, retrieving data from storage 308, and transmitting data to a networked device via network interfaces 312. As discussed more fully with respect to FIG. 4, CPU 310 can receive indications of geo-fence zone exits from geo-fence detector 304 and can communicate with a mobile device using network interfaces 312. In one non-limiting embodiment, CPU 310 can receive location data from GPS receiver 302 and can store the location data in storage 308. In one non-limiting embodiment, storing location data can comprise associated a timestamp with the data. In some non-limiting embodiments, CPU 310 can retrieve location data from GPS receiver 302 according to a pre-defined interval. For example, the pre-defined interval can be once every three minutes. In some non-limiting embodiments, this interval can be dynamically changed based on the estimated length of a walk or the remaining battery life of the device 300. CPU 310 can further be capable of transmitting location data to a remove device or location via network interfaces 312.

FIG. 4 is a flow diagram illustrating a method for tracking a pet according to certain non-limiting embodiments. In step 402, method 400 can be used to monitors the location of a device. In one non-limiting embodiment, monitoring the location of a device can comprise monitoring the GPS position of the device discretely, meaning at regular intervals. For example, in step 402, the wearable device can discretely poll a GPS receiver every five seconds and retrieve a latitude and longitude of a device. Alternatively, in some other non-limiting embodiments, continuous polling of a GPS location can be used. By discretely polling the GPS receiver, as opposed to continuously polling the device, the method can extend the battery life of the mobile device, and reduce the number of network or device resources consumed by the mobile device.

In other non-limiting embodiments, method 400 can utilize other methods for estimating the position of the device, without relying on the GPS position of the device. For example, method 400 can monitor the location of a device by determining whether the device is connected to a known WLAN connection and using the connection to a WLAN as an estimate of the device location. In yet another non-limiting embodiment, a wearable device can be paired to a mobile device via a Bluetooth network. In this embodiment, method 400 can query the paired device to determine its location using, for example, the GPS coordinates of the mobile device.

In step 404, method 400 can include determining whether the device has exited a geo-fence zone. As discussed above, in one non-limiting embodiment, method 400 can include continuously polling a GPS receiver to determine the latitude and longitude of a device. In this embodiment, method 400 can then compare the received latitude and longitude to a known geo-fence zone, wherein the geofenced region includes a set of latitude and longitude points defining a region, such as a polygonal region. When using a WLAN to indicate a location, method 400 can determine that a device exits geo-fence zone when the presence of a known WLAN is not detected. For example, a tracking device can be configured to identify a home network (e.g., using the SSID of the network). When the device is present within the home (e.g., when a pet is present within the home), method 400 can determine that the device has not exited the geo-fence zone. However, as the device moves out of range of the known WLAN, method 400 can determine that a pet has left or exited the geo-fence zone, thus implicitly constructing a geo-fence zone based on the contours of the WLAN signal.

Alternatively, or in conjunction with the foregoing, method 400 can employ a continuous detection method to determine whether a device exits a geo-fence zone. Specifically, WLAN networks generally degrade in signal strength the further a receiver is from the wireless access point or base station. In one non-limiting embodiment, the method 400 can receive the signal strength of a known WLAN from a wireless transceiver. In this embodiment, the method 400 can set one or more predefined thresholds to determine whether a device exits geo-fence.

For example, a hypothetical WLAN can have signal strengths between ten and zero, respectively representing the strongest possible signal and no signal detected. In certain non-limiting embodiments, method 400 can monitor for a signal strength of zero before determining that a device has exited a geo-fence zone. Alternatively, or in conjunction with the foregoing, method 400 can set a threshold signal strength value of three as the border of a geo-fence region. In this example, the method 400 can determine a device exited a geo-fence when the signal strength of a network drops below a value of three. In some non-limiting embodiments, the method 400 can utilize a timer to allow for the possibility of the network signal strength returning above the predefined threshold. In this embodiment, the method 400 can allow for temporary disruptions in WLAN signal strength to avoid false positives and/or short term exists.

If in method 400 the server determines that a wearable device has not exited a geo-fence zone, method 400 can continue to monitor the device location in step 402, either discretely or continuously. Alternatively, if method 400 determines that a device has exited a geo-fence zone, a sensor can send a signal instructing the wearable device to turn on an illumination device, as shown in step 406. The illumination device, for example, can include a light emitting diode (LED) or any other light. The illumination device can be positioned within the housing of the wearable device, and can illuminate at least the top cover of the wearable device, also referred to as a wearable device. In yet another example, the illumination device can light up at least a part and/or a whole surface of the wearable device. In certain non-limiting embodiments, instead of an illumination device the wearable device can include any other indicator, such as a sound device, which can include a speaker, and/or a vibration device. In step 406, therefore, any of the above indicators, whether an illumination device, a sound device, or a vibration device can be turned on or activated.

In certain non-limiting embodiments, a mobile device user can be prompted to confirm whether the wearable device has exited the geo-fence zone. For example, a wearable device can be paired with a mobile device via a Bluetooth connection. In this embodiment, the method 400 can comprise alerting the device via the Bluetooth connection that the illumination device has been turned on, in step 406, and/or that the wearable device has exited the geo-fence zone, in step 404. The user can then confirm that the wearable device has existed the geo-fence zone (e.g., by providing an on-screen notification). Alternatively, a user can be notified by receiving a notification from a server based on the data received from the mobile device.

Alternatively, or in conjunction with the foregoing, method 400 can infer the start of a walk based on the time of day. For example, a user can schedule walks at certain times during the day (e.g., morning, afternoon, or night). As part of detecting whether a device exited a geo-fence zone, method 400 can further inspect a schedule of known walks to determine whether the timing of the geo-fence exiting occurred at an expected walk time (or within an acceptable deviation therefrom). If the timing indicates an expected walk time, a notification to the user that the wearable device has left the geo-fence zone can be bypassed.

Alternatively, or in conjunction with the foregoing, the method 400 can employ machine-learning techniques to infer the start of a walk without requiring the above input from a user. Machine learning techniques, such as feed forward networks, deep forward feed networks, deep convolutional networks, and/or long or short term memory networks can be used for any data received by the server and sensed by the wearable device. For example, during the first few instances of detecting a wearable device exiting the geo-fence zone, method 400 can continue to prompt the user to confirm that they are aware of the location of the wearable device. As method 400 receives either a confirmation or denial from the user, method 400 can train a learning machine located in the server to identify conditions associated with exiting the geo-fence zone. For example, after a few prompt confirmations, a server can determine that on weekdays between 7:00 AM and 7:30 AM, a tracking device repeatedly exits the geo-fence zone (i.e., conforming to a morning walk of a pet). Relatedly, server can learn that the same event (e.g., a morning walk) can occur later on weekends (e.g., between 8:00 AM and 8:30 AM). The server can therefore train itself to determine various times when the wearable device exits the geo-fence zone, and not react to such exits. For example, between 8:00 AM and 8:30 AM on the weekend, even if an exit is detected the server will not instruct the wearable device to turn on illumination device 406.

In certain non-limiting embodiments, the wearable device and/or server can continue to monitor the location and record the GPS location of the wearable device, as shown in step 408. In step 410, the wearable device can transmit location details to a server and/or to a mobile device.

In one non-limiting embodiment, the method 400 can continuously poll the GPS location of a wearable device. In some non-limiting embodiments, a poll interval of a GPS device can be adjusted based on the battery level of the device. For example, the poll interval can be reduced if the battery level of the wearable device is low. In one non-limiting example the poll interval can be reduced from every 3 minutes to every 15 minutes. In alternative embodiments, the poll interval can be adjusted based on the expected length of the wearable device's time outside the geo-fence zone. That is, if the time outside the geo-fence zone is expected to last for thirty minutes (e.g., while walking a dog), the server and/or wearable device can calculate, based on battery life, the optimal poll interval. As discussed above, the length of a walk can be inputted manually by a user or can be determined using a machine-learning or artificial intelligence algorithm based on previous walks.

In step 412, the server and/or the wearable device can determine whether the wearable device has entered the geo-fence zone. If not, steps 408, 410 can be repeated. The entry into the geo-fence zone may be a re-entry into the geo-fence zone. That is, it may be determined that the wearable device has entered the geo-fence zone, having previously exited the geo-fence zone. As discussed above, the server and/or wearable device can utilize a poll interval to determine how frequently to send data. In one non-limiting embodiment, the wearable device and/or the server can transmit location data using a cellular or other radio network. Methods for transmitting location data over cellular networks are described more fully in commonly owned U.S. Non-Provisional application Ser. No. 15/287,544, entitled “System and Method for Compressing High Fidelity Motion Data for Transmission Over a Limited Bandwidth Network,” which is hereby incorporated by reference in its entirety.

Finally, if the server and/or wearable device determine that the wearable device has entered the geo-fence zone, the illumination device, or any other indicated located on the wearable device, can be turned off. In some non-limiting embodiments, not shown in FIG. 4, when a wearable device exits the geo-fence zone the user can choose to turn off the illumination device. For example, when a user of a mobile device confirms that the wearable device has exited the geo-fence zone, the user can instruct the server to instruct the wearable device, or instruct the wearable device directly, to turn off the illumination device.

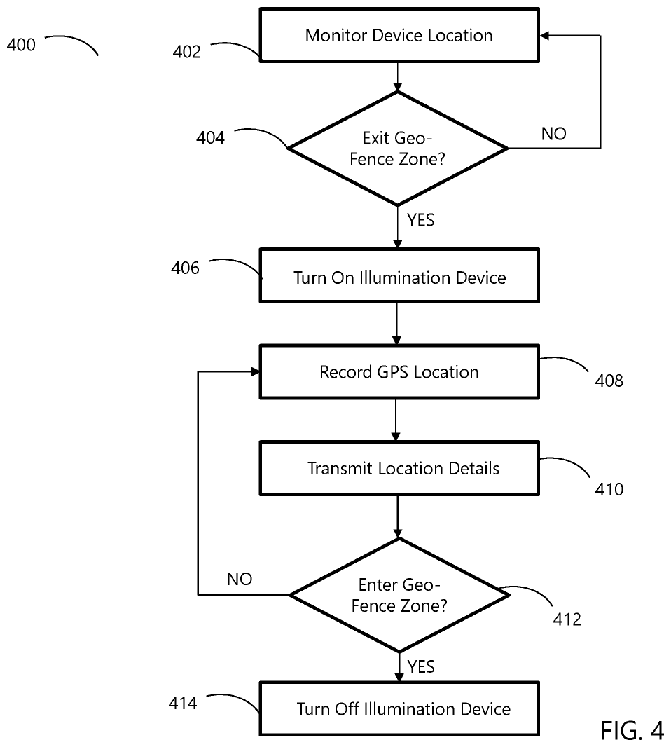

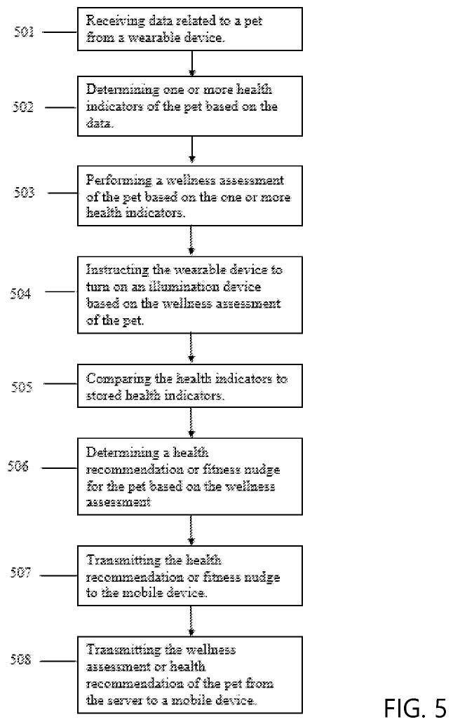

FIG. 5 is a flow diagram illustrating a method for tracking and monitoring the pet according to certain non-limiting embodiments. The steps of the method shown in FIG. 5 can be performed by a server, the wearable device, and/or the mobile device. The wearable device can sense, detect, or collect data related to the pet. The data can include, for example, data related to location or movement of the pet. In certain non-limiting examples, the wearable device can include one or more sensors, which can allow the wearable device to detected movement of the pet. In some non-limiting embodiments, the sensor can be a collar mounted triaxial accelerometer, which can allow the wearable device to detect various body movements of the pet. The various body movement can include, for example, any bodily movement associated with itching, scratching, licking, walking, drinking, eating, sleeping, and shaking, and/or any other bodily movement associated with an action performed by the pet. In certain examples, the one or more sensors can detect a pet jumping around, excited for food, eating voraciously, drinking out of the bowl on the wall, and/or walking around the room. The one or more sensors can also detect activity of a pet after a medical procedure or veterinary visit, such as a castration or ovariohysterectomy visit.

In certain non-limiting embodiments, the data collected via the one or more sensors can be combined with data collected from other sources. In one non-limiting example, the data collected from the one or more sensors can be combined with video and/or audio data acquired using a video recording device. Combining the data from the one or more sensors and the video recording device can be referred to as data preparation. During data preparation, the video and/or audio data can utilize video labeling, such as behavioral labeling software. The video and/or audio data can be synchronized and/or stored along with the data collected from the one or more sensors. The synchronization can include comparing sensor data to video labels, and aligning the sensor data with the video labels to minute, second, or sub-second accuracy. The data can be aligned manually by a user or automatically, such as using a semi-supervised approach to estimate offset. The combined data from the one or more sensors and video recording device can be analyzed using machine learning or any of the algorithms describes herein. The data can also be labeled as training data, validation data, and/or test data.

The data can be sensed, detected, or collect either continuously or discretely, as discussed in FIG. 4 with respect to location data. In certain non-limiting embodiments, the activities of the pet can be continuously sensed or detected by the wearable device, with data being continuously collected, but the wearable device can discretely transmit the information to the server in order to save battery power and/or network resources. In other words, the wearable device can continuously monitor or track the pet, but transmit the collected data every finite amount of time. The finite amount of time used for transmission, for example, can be one hour.

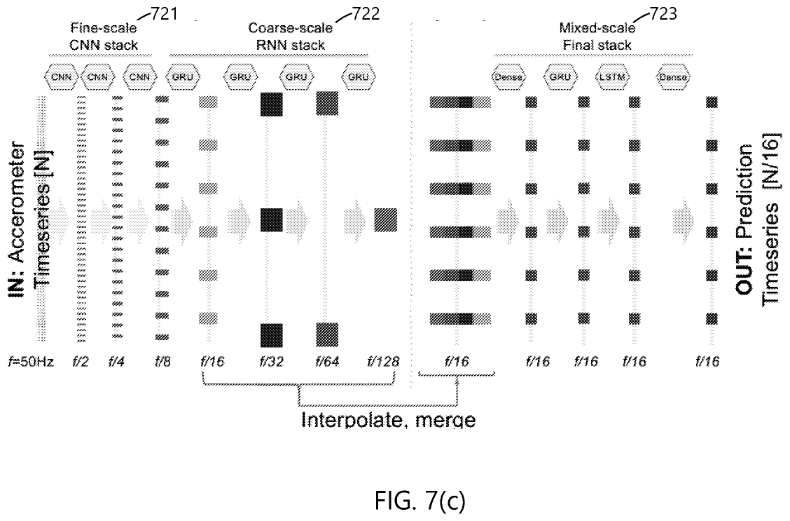

In step 501, the data related to the pet from the wearable device can be received at a server and/or the mobile device of the user. Once received, the data can be processed by the server and/or mobile device to determine one or more health indicators of the pet, as shown in step 502. The server can utilize a machine learning tool, for example, such as a deep neural network using convolutional neural network and/or recurrent neural network layers, as described below. The machine learning tool can be referred to as an activity recognition algorithm or model. In certain non-limiting embodiments, the machine learning tool can include one or more layer modules as shown in FIG. 7. Using this machine learning tool, health indicators, also referred to as behaviors of the pet wearing the device, can be determined. The one or more health indicators comprise a metric for itching, scratching, licking, walking, drinking, eating, sleeping, and shaking. The metric can be, for example, the distance walked, time slept, and/or an amount of itching by a pet.

The machine learning tool can be trained. To train the machine learning tool, for example, the server can aggregate data from a plurality of wearable devices. The aggregation of data from a plurality of wearable devices can be referred to as crowd-sourcing data. The collected data from one or more pets can be aggregated and/or classified in order to learn one or more trends or relationships that exist in the data. The learned trends or relationships can be used by the server to determine, predict, and/or estimate the health indicators from the received data. The health indicators can be used for determining any behaviors exhibited by the pet, which can potentially impact the wellness or health of the pet. Machine learning can also be used to model the relationship between the health indicators and the potential impact on the health or wellness of the pet. For example, the likelihood that a pet can be suffering from an ailment or set of ailments, such as dermatological disorders. The machine learning tool can be automated and/or semi-automated. In semi-automated models, the machine learning can be assisted by a human programmer that intervenes with the automated process and helps to identify or verify one or more trends or models in the data being processed during the machine learning process.

In certain non-limiting embodiments, the machine learning tool used to convert the data, such as time series accelerometer readings, into predicted health indicators can use windowed methods that predict behaviors for small windows of time. Such embodiments can produce a single prediction per window. On the other hand, other non-limiting embodiments rather than using small windows of time, and data included therein, the machine learning tool can run on an aggregated amount of data. The data received from the wearable device can be aggregated before it can be fed into the machine learning tool, thereby allowing an analysis of a great number of data points. The aggregation of data, for example, can break the data points which are originally received at a frequency window of 3 hertz, into minutes of an hour, hour of a day, day of week, month of year, or any other periodicity that can ease the processing and help the modeling of the machine learning tool. When the data is aggregated more than once, there can be a hierarchy established on the data aggregation. The hierarchy can be based on the periodicity of the data bins in which the aggregated data are placed, with each reaggregation of the data reducing the number of bins into which the data can be placed.

For example, 720 data points, which in some non-limiting embodiments would be processed individually using small time windows, can be aggregated into 10 data points for processing by the machine learning tool. In further examples, the aggregated data can be reaggregated into a smaller number of bins to help further reduce the number data points to be processed by the machine learning tool. By running on an aggregated amount of data can help to produce a large number of matchings and/or predictions. The other non-limiting embodiments can learn and model trends in a more efficient manner, reducing the amount of time needed for processing and improving accuracy. The aggregation hierarchy described above can also help to reduce the amount of storage. Rather than storing raw data or data that is lower in the aggregation hierarchy, certain non-limiting embodiments can store data in a high aggregation hierarchy format.

In some other embodiments, the aggregation can occur after the machine learning process using the neural network, with the data merely being resampled, filtered, and/or transformed before it is processed by the machine learning tool. The filtering can include removing interference, such as brown noise or white noise. The resampling can include stretching or compressing the data, while the transformation can include flipping the axes of the received data. The transformation can also exploit natural symmetry of the data signals, such as left/right symmetry and different collar positions. In some non-limiting embodiments, data augmentation can include adding noise to the signal, such as brown, pink, or white noise.

In step 503, a wellness assessment of the pet based on the one or more health indicators can be performed. The wellness assessment, for example, can include an indication of one or more diseases, health conditions, and/or any combination thereof, as determined and/or suggested by the health indicators. The health conditions, for example, can include one or more of: a dermatological condition, an ear infection, arthritis, a cardiac episode, a tooth fracture, a cruciate ligament tear, a pancreatic episode and/or any combination thereof. In certain non-limiting embodiments, the server can instruct the wearable device to turn on an illumination device based on the wellness assessment of the pet, as shown in step 504. In step 505, the health indicator can be compared to one or more stored health indicators, which can be based on previously received data. If a threshold different is detected by comparing the health indicator with the stored health indicator, the wellness assessment can reflect such a detection. For example, the server can detect that the pet is sleeping less by a given threshold, itching more by a given threshold, of eating less by a given threshold. Based on these given or preset thresholds, a wellness assessment can be performed. In some non-limiting embodiments, the thresholds can also be determined using the above described machine learning tool. The wellness assessment, for example, can identify that the pet is overweight or that the pet can potentially have a disease.

In step 506, the server can determine a health recommendation or fitness nudge for the pet based on the wellness assessment. A fitness nudge, in certain non-limiting embodiments, can be an exercise regimen for a pet. For example, a fitness nudge can be having the pet walk a certain number of steps per day and/or run a certain number of steps per day. The health recommendation or fitness nudge, for example, can provide a user with a recommendation for treating the potential wellness or health risk to the pet. Health recommendation, for example, can inform the user of the wellness assessment and recommend that the user take the pet to a veterinarian for evaluation and/or treatment, or can provide specific treatment recommendations, such as a recommendation to feed pet a certain food or a recommendation to administer an over the counter medication. In other non-limiting embodiments, the health recommendation can include a recommendation for purchasing one or more pet foods, one or more pet products and/or any combination thereof. In steps 507 and 508, the wellness assessment, health recommendation, fitness nudge and/or any combination thereof can be transmitted from the server to the mobile device, where the wellness assessment, the health recommendation and/or the fitness nudge can be displayed, for example, on a graphic user interface of the mobile device.

In some non-limiting embodiments, the data received by the server can include location information determined or obtained using a GPS. The data can be received via a GPS received at the wearable device and transmitted to the server. The location data can be used similar to any other data described above to determine one or more health indicators of the pet. In certain non-limiting embodiments, the monitoring of the location of the wearable device can include identifying an active wireless network within a vicinity of the wearable device. When the wearable device is within the vicinity of the wearable device, the wearable device can be connected to the wireless network. When the wearable device has exited the geo-fence zone, the active wireless network can no longer be in the vicinity of the wearable device. In other embodiments, the geo-fence can be predetermined using latitude and longitudinal coordinates.

Certain non-limiting embodiments can be directed to a method for data analysis. The method can include receiving data at an apparatus. The data can include at least one of financial data, cyber security data, electronic health records, acoustic data, human activity data, or pet activity data. The method can also include analyzing the data using two or more layer modules. Each of the layer modules includes at least one of a many-to-many approach, striding, downsampling, pooling, multi-scaling, or batch normalization. In addition, the method can include determining an output based on the analyzed data. The output can include a wellness assessment, a health recommendation, a financial prediction, or a security recommendation. The two or more layers can include at least one of full-resolution convolutional neural network, a first pooling stack, a second pooling stack, a resampling step, a bottleneck layer, a recurrent stack, or an output module. In some embodiment, the determined output can be displayed on a mobile device.

As described in the example embodiments shown in FIG. 5, the data can be received, processed, and/or analyzed. In certain non-limiting embodiments, the data can be processed using a time series classification algorithm. Time series classification algorithms can be used to assess or predict data over a given period of time. An activity recognition algorithm that tracks a pet's moment-to-moment activity over time can be an example of a time series classification algorithm. While some time series classification algorithms can utilize K-nearest neighbors and support vector machine approaches, other algorithms can utilize deep-learning based approaches, such as those examples described below.

In certain non-limiting embodiments, the activity recognition algorithm can utilize machine learning models. In such embodiments, an appropriate time series can be acquired, which can be used to frame the received data. Hand-crafted statistical and/or spectral feature vectors can then be calculated over one or more finite temporal windows. A feature can be an individual measurable property or characteristic being observed via the wearable device. A feature vector can include a set of one or more features. Hand-crafted can refer to those feature vectors derived using manually predefined algorithms. A training model, such as K-nearest neighbor (KNN), naïve Bayes (NB), decision trees or random forests, support vector machine (SVM), or any other known training model, can map the calculated feature vectors to activity predictions. The training model can be evaluated on new or held-out time series data to infer activities.

One or more training models can be used or integrated to improve prediction outcomes. For example, an ensemble-based method can be used to integrate one or more training models. Collective of Transformation-based Ensembles (COTE) and the hierarchal voting variant HIVE-COTE are examples of ensemble-based methods.

Rather than using machine learning models or tools, such as KNN, NB, or SVM, some other embodiments can utilize one or more deep learning or neural-network models. Deep learning or neural-network models do not rely on hand-crafted feature vectors. Instead, deep learning or neural-network models use learned feature vectors derived from a training procedure. In certain non-limiting embodiments, neural networks can include computational graphs composed of many primitive building blocks, with each block performing a weighted sum of it inputs and introducing a non-linearity. In some non-limiting embodiments, a deep learning activity recognition model can include a convolutional neural network (CNN) component. While in some examples a neural network can train a learned weight for every input-output pair, CNNs can convolve trainable fixed-length kernels or filters along their inputs. CNNs, in other words, can learn to recognize small, primitive features (low levels) and combine them in complex ways (high levels).

In certain non-limiting embodiments, pooling, padding, and/or striding can be used to reduce the size of a CNN's output in the dimensions that the convolution is performed, thereby reducing computational cost and/or making overtraining less likely. Striding can describe a size or number of steps with which a filter window slides, while padding can include filling in some areas of the data with zeros to buffer the data before or after striding. Pooling, for example, can include simplifying the information collected by a convolutional layer, or any other layer, and creating a condensed version of the information contained within the layers. In some examples, a one-dimensional (1-D) CNN can be used to process fixed-length time series segments produced with sliding windows. Such 1-D CNN can run in a many-to-one configuration that utilizes pooling and striding to concatenate the output of the final CNN layer. A fully connected layer can then be used to produce a class prediction at one or more time steps.

As opposed to 1-D CNNs that convolve fixed-length kernels along an input signal, recurrent neural networks (RNNs) process each time step sequentially, so that an RNN layer's final output is a function of every preceding timestep. In certain non-limiting embodiments, an RNN variant known as long short-term memory (LSTM) model can be used. LSTM can include a memory cell and/or one or more control gates to model time dependencies in long sequences. In some examples the LSTM model can be unidirectional, meaning that the model processes the time series in the order it was recorded or received. In another example, if the entire input sequence is available two parallel LSTM models can be evaluated in opposite directions, both forwards and backwards in time. The results of the two parallel LSTM models can be concatenated, forming a bidirectional LSTM (bi-LSTM) that can model temporal dependencies in both directions.

In some non-limiting embodiments, one or more CNN models and one or more LSTM models can be combined. The combined model can include a stack of four unstrided CNN layers, which can be followed by two LSTM layers and a softmax classifier. A softmax classifier can normalize a probability distribution that includes a number of probabilities proportional to the exponentials of the input. The input signals to the CNNs, for example, are not padded, so that even though the layers are unstrided, each CNN layer shortens the time series by several samples. The LSTM layers are unidirectional, and so the softmax classification corresponding to the final LSTM output can be used in training and evaluation, as well as in reassembling the output time series from the sliding window segments. The combined model though can operate in a many-to-one configuration.

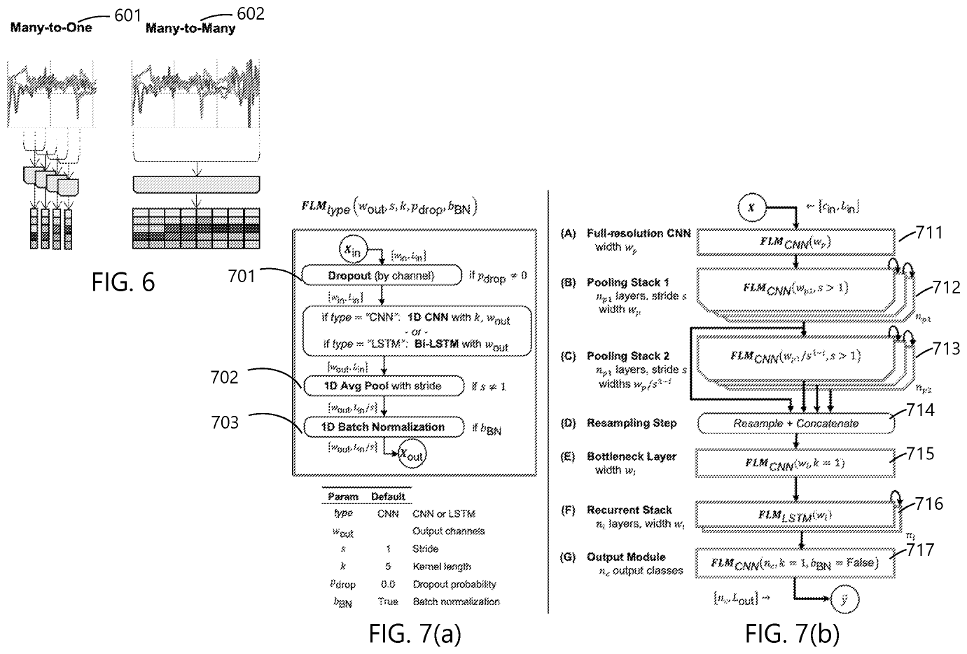

FIG. 6 illustrates an example of two deep learning models according to certain non-limiting embodiments. In particular, FIG. 6 illustrates a many-to-one model 601 and a many-to-many model 602. In a many-to-one approach or model 601 an input can first be divided into fixed-length overlapping windows. The model can then process each window individually, generate a class prediction for each window, and the predictions can be concatenated into an output time series. The many-to-one model 601 can therefore be evaluated once for each window. In a many-to-many model 602, on the other hand, the entire output time series can be generated with a single model evaluation. A many-to-many model 602 can be used to process the one or more input signals at once, without requiring sliding, fixed-length windows.

In certain non-limiting embodiments, a model can incorporate features or elements taken from one or more models or approaches. Doing so can help to improve the accuracy of the model, prevent bias, improve generalization, and allow for faster processing of data. Using elements from a many-to-many approach can allow for processing of the entire input signal, which may include one or more signals. In some non-limiting embodiments the model can also include striding or downsampling. Each layer of the model can use striding to reduce the number of samples that are outputted after processing. Using striding or downsampling can help to improve computational efficiency and allow subsequent layers to model dynamics over longer time ranges. In certain non-limiting embodiments the model can also utilize multi-scaling, which can help to downsample beyond the output frequency to model longer-range temporal dynamics.

A model that utilizes features or elements of many-to-many models, striding or downsampling, auto-scaling, and multi-scaling can allow the model to be applied to a time series of arbitrary length. For example, the model can infer an output time series of length proportional to the input length. Using features or elements of many-to-many model, which can be referred to as a sequence-to-sequence model, can allow the model to not be tied to the length of its input. Further, in some examples, a larger model would not be needed for a larger time series length or sliding window length.

In certain non-limiting embodiments the model can include a stack of parameterized modules, which can be referred to as flexible layer modules (FLMs). One or more FLMs can be combined into signal-processing stacks and can be tweaked and re-configured to train efficiently. Each FLM can be coverage-preserving, meaning that the input and/or output of an FLM can differ in sequence length due to a stride ratio, and/or the time period that the input and output cover can be identical. FLM can be represented using the following notation: FLM_{type}(wₒᵤₜ, S=k=5, p_{drop}=0.0). type can represent the type of the primary trainable sub-layer (‘cnn’ for a 1-D CNN or ‘lstm’ for a bi-directional LSTM). wₒᵤₜ can be the number of output channels (the number of filters for a cnn or the dimensionality of the hidden state for an lstm). s can represent a stride ratio (default 1), while k can represent the kernel length (for CNNs, default 5), and p_{drop} represents the dropout probability (default 0.0). In certain non-limiting embodiments, when s>1 then a 1-D average-pooling with stride s and pooling kernel length s reduces the output length by a factor of s.

Each FLM can include a dropout layer which can randomly drop out sensor channels during training with probability p_{drop}. The dropout layer can be with a 1D CNN or a bidirectional LSTM layer. A 1D average-pooling layer which pools and strides the output of the CNN or LSTM layer whenever s does not equal zero. The 1D average-pooling layer can be referred to as a strided layer, and can include a matching pooling step so that all CNN or LSTM output samples are represented in the FLM output. A batch normalization (b_{BN}) layer can also be included in the FLM layer. The batch layer and/or the dropout layer can serve to regularize the network and improve training dynamics.

In certain non-limiting embodiments, a CNN layer can be configured to zero-pad the input by ceil

$$( \frac{k - 1}{2} ) ,$$

so that the input and output signal lengths are equal. Each FLM can therefore map an input tensor Xᵢₙ of size [wᵢₙ, Lᵢₙ] to an output tensor Xᵢₙ of size [wₒᵤₜ, Lₒᵤₜ=Lᵢₙ/s]. In some non-limiting embodiments other modifications can be added, such as one or more gated recurrent unit (GRU) layers, which can include a gating mechanism in recurrent neural networks. Other modifications can include grouping of CNN filters, and different strategies for pooling, striding, and/or dilation.

FIGS. 7(a) and 7(b) illustrate a model architecture according to certain non-limiting embodiments. In particular, FIG. 7(a) illustrates an embodiment of a model that includes one or more stack of FLM, such as a dropout layer 701, a 1D average-pooling 702, and a 1D batch normalization 703, as described above. FIG. 7(b) illustrates a component architecture, in which one or more FLMs can be grouped into components that can be parameterized and combined to implement time series classifiers of varying speed and complexity. The component architecture, for example, can include one or more components or FLMs, such as full-resolution CNN 711, first pooling stack 712, second pooling stack 713, bottleneck layer 715, recurrent stack 716, and/or output module 717. The component architecture can also include resampling step 714. Any of the one or more components or FLMs shown in FIG. 7(b) can be removed.

Full-resolution CNN 711 can include high resolution processing characterized as s=1 and type=CNN. In certain non-limiting embodiments, full-resolution CNN 711 can be a CNN filter which can process the input signal without striding or pooling, to extract information at the finest available temporal resolution. This layer can be computationally expensive, in some non-limiting embodiments, because it can be applied to the full-resolution input signal. First pooling stack 712 can be used to downsample from the input to the output frequency, characterized as s>1 and type=CNN. Stack 712 of nₚ₁ CNN modules (each strided by s) downsamples the input signal by a total factor of sⁿ^{ₚ₁}. nₚ₁ can be the number of CNN modules included within first pooling stack 712. The output length of stack 712 can be determined using an output stride ratio, sₒᵤₜ=sⁿ^{ₚ₁}, and thus the output length of the network for a given input, L_{OUT}=L_{IN}/sⁿ^{ₚ₁}. With L_{OUT} being the output length and L_{IN} being the input length.

Second pooling stack 713 can be used to further downsample the signal, and can be characterized as s>1 and type=CNN. This stack of nₚ_{₂} modules, each strided by s, further downsamples the output of the previous layer, beyond the output frequency, in order to capture slower temporal dynamics. To protect against overtraining, the width of each successive module can be reduced by a factor of s so that wᵢ=wₚs¹⁻ⁱ for i=1 . . . nₚ_{₂}. A resampling step 714 can also be used to process the signal. In this step, the output of second pooling stack 713 can be resampled via linear interpolation to match the network output length L_{OUT}. These outputs can be concatenated with the final module output of first pooling stack 712. Without resampling step 713, the lengths of the outputs of second pooling stack 713 cannot match the output length, and cannot be processed together in the next layer. Exposing each intermediate output of second pooling stack 713 using resampling step 714, as opposed to only exposing the final output of second pooling stack 713, can help to improve the model's training dynamics and accuracy.

The model can also include bottleneck layer 715, which can effectively reduce the width of the concatenated outputs from resampling step 714. In other words, bottleneck layer 715 can help to minimize the number of learned weights needed in recurrent stack 716. This bottleneck layer can allow a large number of channels to be concatenated from second pooling stack 713 and resampling step 714 without resulting in overtraining or excessively slowing down the network. As a CNN with kernel length k=1, bottleneck layer 715 can be similar to a fully connected dense network applied independently at each time step.

Recurrent stack 716 can be characterized as s=1 and type=LSTM. In certain non-limiting embodiments, recurrent stack 716 can include nₗ recurrent LSTM modules. Stack 716 provides for additional capacity that allows modeling of long-range temporal dynamics and/or improves the output stability of the network. Output module 717 provides predictions for each output time step and can be characterized using s=1 and k=1. As with bottleneck layer 715, output module 717 can be implemented as a CNN with k=1. In certain non-limiting embodiments, multi-class outputs can be achieved using a softmax activation function, which converts and normalizes the layer outputs zᵢ to be the class probability distribution P(z)ᵢ according to the formula P(z)ᵢ=e^{z}^{ᵢ}/Σe^{z}^{ⱼ}. One or more layers 711-717 can be independently reconfigured or removed to optimize the model's properties.

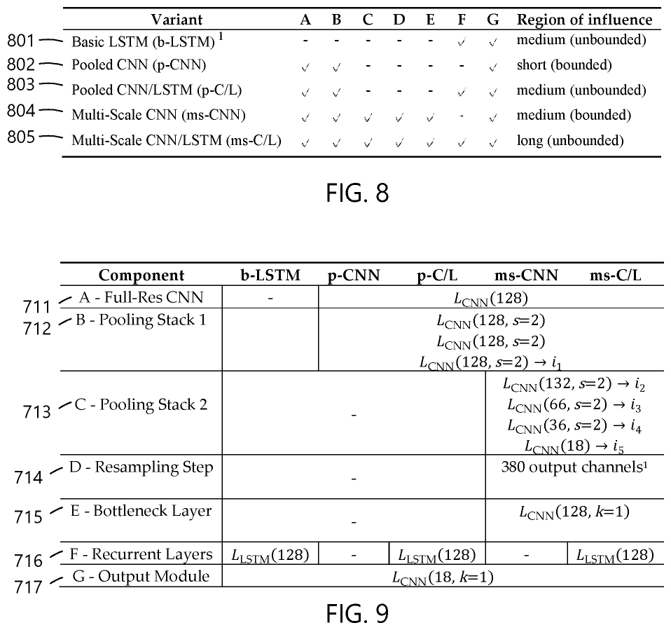

FIG. 8 illustrates examples of a model according to certain non-limiting embodiments. In particular, FIG. 8 illustrates a variant to the model shown in FIG. 7(b). Basic LSTM (b-LSTM) model 801 can merely include a recurrent stack 716 and output module 717. In other words, b-LSTM does not downsample the network input, and instead includes one or more FLM_{LSTM} layers followed by an output module. Pooled CNN (p-CNN) model 802 can include full-resolution CNN 711, first pool stack 712, recurrent stack 716, and output module 717. p-CNN model 802 can therefore be a stack of FLM_{CNN} layers where one or more of the layers is strided, so that the output frequency is lower than the input frequency. Model 802 can improve computational efficiency and increase the timescales that the network can model, relative to an unstrided CNN stack.

Pooled CNN or LSTM model 803 (p-C/L) can include full-resolution CNN 711, first pool stack 712, recurrent stack 716, and output module 717. p-C/L can add one or more recurrent layers that operate at the output frequency immediately before the output modules layer. Multi-scale CNN (ms-CNN) 804 can include full-resolution CNN 711, first pooling stack 712, second pooling stack 713, resample step 714, bottleneck layer 715, and/or output module 717. Multi-scale CNN or LSTM (ms-C/L) 805 can include full-resolution CNN 711, first pooling stack 712, second pooling stack 713, resample step 714, bottleneck layer 715, recurrent stack 716, and/or output module 717. ms-CNN and ms-C/L variants modify the p-CNN and p-C/L variants by adding a second pooling stack and subsequent resampling and bottleneck layers. This progression from p-CNN to ms-C/L demonstrates the effect of increasing the variants' ability to model long-range temporal interactions, both through additional layers of striding and pooling, as well as through recurrent LSTM layers.

A dataset can be used to test the effectiveness of the model. For example, the Opportunity Activity Recognition Dataset can be used to test the effectiveness of the model shown in FIG. 7(b). The Opportunity Activity Recognition Dataset can include six hours of recordings of several subjects using a diverse array of sensors and labels, such as seven inertial measurement units (IMU) with accelerometer, gyroscope, and magnetic sensors, and twelve Bluetooth accelerometers. See Daniel Roggen et al., “Collecting Complex Activity Data Sets in Highly Rich Networked Sensor Environments,” Seventh International Conference on Networked Sensing Systems (INSS '10), Kassel, Germany, (2010), available at https://archive.ics.uci.edu/ml/datasets/opportunity+activity+recognition. The Opportunity Activity Recognition Dataset is hereby incorporated by reference in its entirety. Each subject was recorded performing a practice session of predefined and scripted activities, as all as five sessions in which the subject performed the activities of daily living in an undefined order. The dataset can be provided at a 30 or 50 Hz frequency. In some examples linear interpolation can be used to fill-in missing sensor data. In addition, in certain non-limiting embodiments instead of rescaling and clipping all channels to a [0,1] interval using a predefined scaling, the data can be rescaled to have zero mean and unit standard deviation according to the statistics of the training set.

FIG. 7(c) illustrates a model architecture according to certain non-limiting embodiments. In particular, the embodiment in FIG. 7(c) shows a CNN model that processes accelerator data in a single shot. The first seven layers, which include fine-scale CNN 721 and coarse-scale RNN stack, can each decimate the signal twice to model increasingly long-range effects. The final four layers, which include mixed-scale final stack 723, can combine outputs from various scales to yield predictions. For example, the data from coarse-scale RNN stack 722 can be interpolated and merged at a frequency of 50 Hz divided by 16.

The six model variants shown in FIG. 8 can vary widely in their implementation due to the number of layers in each component and/or the configuration of each channel, such as striding and number of output layers. Therefore, in the examples provided below a specific reference architecture for each variant can be tested. The model parameters remain consistent, or slightly varied, in order to accurately compare the six variants. FIG. 9 illustrates an example embodiment of the models shown in FIG. 8 and the layer modules shown in FIG. 7. Specifically, the number, size, and configuration of each layer for the tested models can be seen in FIG. 9.

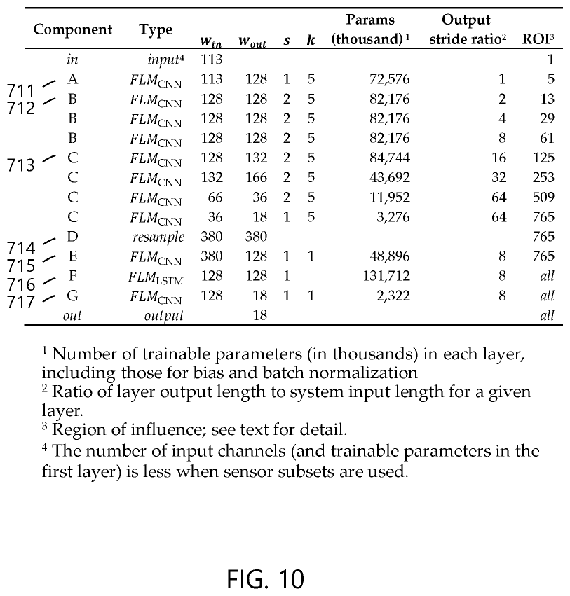

FIG. 10 illustrates an example architecture of one or more of the models shown in FIG. 8 and the layer modules shown in FIG. 7. In particular, the model illustrates a more detailed architecture of ms-C/L shown in FIG. 8. A region of influence (ROI) for a given layer can refer to the maximum number of input samples that can influence the calculation of an output of a given layer. The ROI can be increased by larger kernels, by larger stride ratios, and/or by additional layers, and can represent an upper limit on the timescales that an architecture is capable of modeling. In some example, the ROI can be calculated for CNN-type layers, since the region of influence of bi-directional LSTMs can be the entire input. The ROIᵢ for a FLM_{CNN}(sᵢ, kᵢ) layer i that is preceded in a stack only by other FLM_{CNN} layers can be calculated by using the following equation: ROIᵢ=ROIᵢ₋₁+(kᵢ−1)Σⱼ₌₁ⁱsⱼ.

As described above, in certain non-limiting embodiments, such as those shown in FIGS. 7(a) and 7(b), the model can be a many-to-many model used to process signals of any length. In some non-limiting embodiments, however, due to memory, efficiency, and latency constraints, it can be helpful to divide long input signals into segments using a sliding window of a moderate fixed length and with some segment overlap. The sliding window, for example, can have a length of 512 samples. It can be helpful to process the segments in batches sized appropriately for available memory, and/or reconstruct the corresponding output signal from the processed segments. In certain other embodiments the signal can be manipulated to avoid edge effects at the start and end of each segment. For example, overlap between the segment can allow these edge regions to be removed without creating gaps in the output signal. The overlap can be 50%, or any other number between 0 to 100%. In yet another example, to prevent signal discontinuities, segments can be averaged using a weighted window, such as a Hanning window, that can de-emphasize the edge regions.

In certain non-limiting embodiments, validation and test set performance can be calculated using both sample-based and event-based metrics. Sample-based metrics can be aggregated across all class predictions, which cannot be affected by the order of predictions. Event-based metrics can be calculated after the output is segmented into discrete events, which can be strongly affected by the order of predictions. Sample-based precision, recall, and F1 scores can be calculated for each output class, including a null class. The F1 score takes into account both precision and recall, and can be calculated as

$$F 1 = 2 \frac{Precision \cdot Recall}{Precision + Recall} .$$

The overall model performance can be summarized, for example, as either a mean F1 score averaged across the non-null classes (F₁ₘ), or as a weighted F1 score (F_{1w}), across all classes, where each class is weighted according to its sample proportion in the ground-truth label set. In other embodiments, a non-null weighted F1 score (F_{1w,nn}) which ignores the null class can be used. For event-based metrics, for example, to condense these extensive metrics into a single figure suitable for summarizing a model's overall performance, an event F1 metric (F₁ₑ). In some non-limiting embodiments, F₁ₑ can be calculated in terms of true positives (TP), false positives (FP), and false negatives (FN). The equation for calculating F₁ₑ can be represented as follows:

$${F}_{1 e} = 2 \frac{Precision \cdot Recall}{Precision + Recall} = 2 \frac{\frac{T P}{T P + F P} \cdot \frac{T P}{T P + F N}}{\frac{T P}{T P + F P} + \frac{T P}{T P + F N}} .$$

TP events can be correct (C) events, while FN events can be incorrect actual events, and FP events can be incorrect returned events. To calculate the overall F₁ₑ, certain non-limiting embodiments can simply sum the TP, FP, and FN counts across all classes. This score can be weighted by event length, meaning that long events can have the same influence as short events. Training speed of the model can be measured as the total time taken to train the model on a given computer, and inference speed of the model can be measured as the average time taken to classify each input sample on a computing system that is representative of the systems on which the model will be most commonly run.

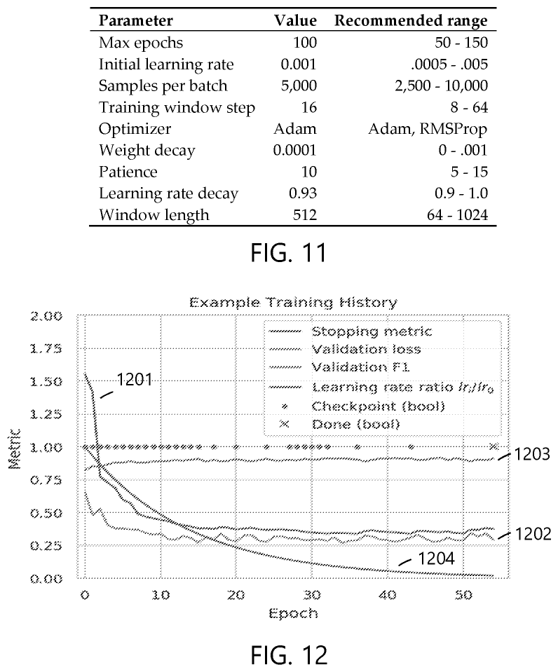

FIG. 11 illustrates an example of model parameters according to certain non-limiting embodiments. In particular, FIG. 11 describes parameters that can be used to train models on GPUs. For example, the parameters can include max epochs, initial learning rate, samples per batch, training window step, optimizer, weight decay, patience, learning rate decay, and/or window length. The training and validation sets can be divided into segments using a sliding window. The window lengths can be integer multiples of the models' output stride ratios to minimize implementation complexity. Because window length can be varied in some testing, the batch size can be adjusted to hold the number of input samples in a batch that can be constant or approximately constant.

In certain non-limiting embodiments, validation loss can be used as an early stopping metrics. However, in some non-limiting embodiments the validation loss can be too noisy to use as an early stopping metric due to the small number of subjects and runs in the validation set. Instead of validation loss, certain non-limiting embodiments can use a customized stopping metric that is more robust, and which penalizes oscillations in performance. The customized stopping metrics can help to limit the model from stopping until model performance can be stabilized. A smoothed validation metric can be determined using an exponentially weighted moving average (with a half-life of 3 epochs) of lᵥ/F_{1w,v}, where lᵥ can be the validation loss, and F_{1w,v} can be the weighted F1 score of the validation set, calculated after each training epoch. The smooth validation metric decreases as the loss and/or the F1 score improve. An instability metric can also be calculated as a standard deviation, average, or median of the past five lᵥ/F_{1w,v} values. The smoother validation metric and the customized stopping metric can be summed to yield a checkpoint metric. The model is checkpointed whenever the checkpoint metric reaches a new minimum, and/or training can be stopped after patience epochs without checkpointing.

FIG. 12 illustrates an example of a representative model training run according to certain non-limiting embodiments. In particular, FIG. 12 illustrates the training history for a ms-C/L model using the parameters shown in FIG. 11. Stopping metric 1201, validation loss 1202, validation F1 1203, and learning rate ratio 1204 are shown in FIG. 12. For example, while the validation loss oscillates and has near-global minima at epochs 15, 24, 34, 41, and 45, the custom stopping metric can adjust more predictably to a minimum at epoch 43. Training can be stopped at epoch 53, and the model from epoch 43 can be restored and used for subsequent inference.

In certain non-limiting embodiments ensembling can be performed using multiple learning algorithms. Specifically, n-fold ensembling can be performed by performing one or more of the following steps: (a) combining the training and validation sets into a single contiguous set; (b) dividing that set into n disjoint folds of contiguous samples; (c) training n independent models where the i^{th} model uses the i^{th} fold for validation and the remaining n−1 folds for training; and (d) ensembling the n models together during inference by simply averaging the outputs before the softmax function is applied. In some non-limiting embodiments, to improve efficiency, the evaluation and ensembling of the n models can be performed using a single computation graph.

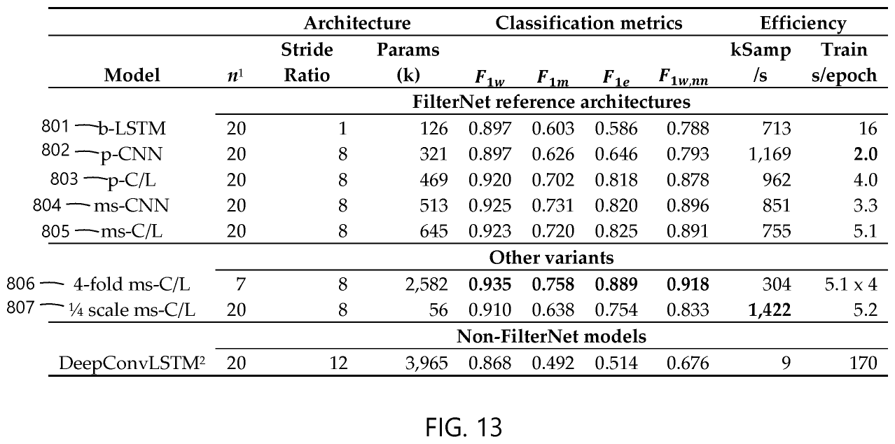

FIG. 13 illustrates performance of example models according to certain non-limiting embodiments. In particular, FIG. 13 shows performance metrics of b-LSTM 801, p-CNN 802, p-C/L 803, ms-CNN 804, and ms-C/L 805. Further, FIG. 13 also shows performance of two variants of ms-C/L 805, such as a 4-fold ensemble of ms-C/L 806 and a ¼ scaled version 807 in which the wow values were scaled by a fourth. 4-fold ms-C/L model 806 can be more accurate than other variants. Other fold variants, such as 3-to-5-fold ms-C/L ensembles, can perform well on many tasks and datasets, especially when inference speed and model size are less important than other metrics.

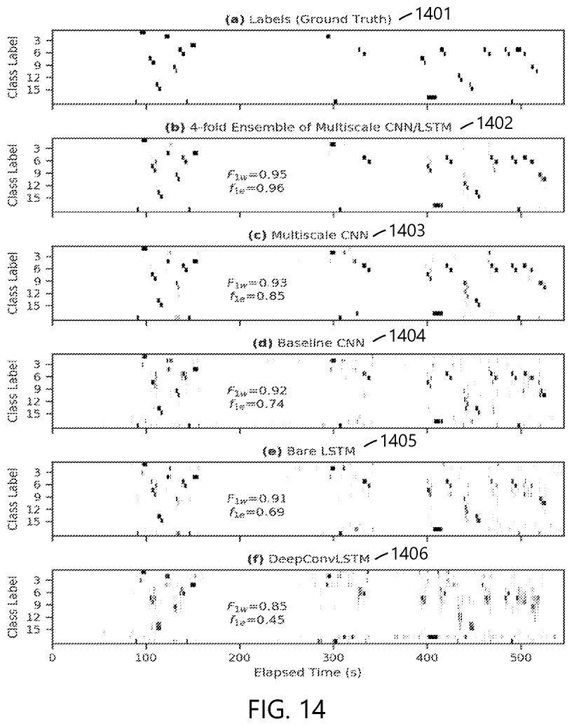

FIG. 14 illustrates a heatmap showing performance of a model according to certain non-limiting embodiments. The heatmaps shown in FIG. 14 demonstrate differences between model outputs, such as labels (ground truth, which in FIG. 14 are provided in the Opportunity Benchmark Dataset) 1401, 4-fold ensemble of multiscale CNN/LSTM 1402, multiscale CNN 1403, baseline CNN 1404, bare LSTM 1405, and deepcovLSTM 1406. In particular, FIG. 14 illustrates ground-truth labels 1401 and model predictions for the first half of the first run in the standard opportunity test set for several models. One or more of the models, such as the ms-C/L architecture, produce fewer short, spurious events. This can help to reduce the false positive count, while also preventing splitting of otherwise correct events. For instance, in the region of interest shown, the event-based F₁ₑ metric increases from 0.85 in multiscale CNN 1403 to 0.96 in 4-fold ensemble of multiscale CNN/LSTM 1402, while the sample-by-sample F_{1w} metric increases only from 0.93 to 0.95. The eventing performance that the one or more models achieves can obviate the need for further event processing and downselection.

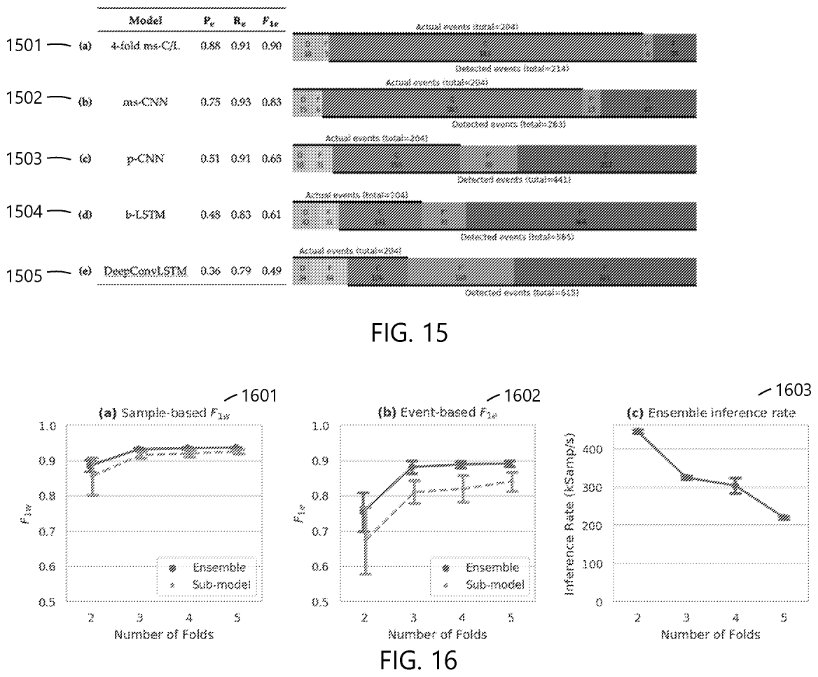

FIG. 15 illustrates performance metrics of a model according to certain non-limiting embodiments. In particular, FIG. 15 shows the results for the same models shown in FIG. 14, but calculated for the entire test set. The event-based metrics shown in FIG. 15 are event-based precision Pₑ, recall Rₑ, F1 score F₁ₑ, and event summary diagrams, each for a single representative run. The event summary diagrams compare the ground truth labels (actual events) to model predictions (detected events). Correct events (C), in certain non-limiting embodiments, can indicate that there is a 1:1 correspondence between actual and detected events. The event summary diagrams depict the number of actual events that are missed (D—deleted) or multiply detected (F—fragmented), as well as the detected fragments (F′—fragmenting) and any spurious detections (I′—insertions).

As shown in the results of FIG. 15, the lower performing models p-CNN 1503, b-LSTM 1504, and/or DeepConvLSTM 1505 suffer from low precision. b-LSTM 1504 detected 131 out of 204 events correctly and generated 434 spurious or fragmented events. ms-CNN model 1502 demonstrates the effect of adding additional strided layers to p-CNN model 1503, which increases the model's region of influence from 61 to 765 samples, meaning that ms-CNN model 1502 can model dynamics occurring over a 12× longer region of influence. The 4×ms-C/L ensemble 1501 can be improved further by adding an LSTM layer, and by making it difficult for a single model to register a spurious event without agreement from the other ensembled models. DeepConvLSTM model 1505 also includes an LSTM layer, but its ROI can be limited to the input window length of 24 samples, which is approximately 3% as long as the ms-C/L ROI. In certain non-limiting embodiments the hidden state of the LSTM at one windowed segment cannot impact the next windowed segment.

FIG. 16 illustrates performance of an n-fold ensembled ms-C/L model according to certain non-limiting embodiments. In particular, FIG. 16 shows sample based F_{1w} 1601 and event based F₁ₑ 1602 weighted F1 metrics. Both F_{1w} and F₁ₑ improve with the number of ensembled models plateauing between 3-5 folds. Ensemble interference rate 1603, however, decreases as the number of folds increase. The effects of model ensembling on accuracy, such as sample-by-sample F_{1w} 1601, event-based F₁ₑ 1602, and inference rate 1603 of the ensemble are plotted in FIG. 16. As described above, the models can be trained on n−1 folds, with the remaining fold used for validation. The 2-fold models, in certain non-limiting embodiments, can therefore have validation sets equal in size to their test sets, and the train and validation sets can simply be swapped in the two sub-models. The higher-n models experience a train-validation split, which can be approximately 67%:33%, 75%:25%, and 80%:20% for the 3, 4, and 5-fold ensembles, respectively. In some non-limiting embodiments, as shown in FIG. 16, event-based metrics 1602 can benefit more from ensembling than sample-by-sample metrics 1601, as measured by the difference between the ensemble and sub-model metrics.

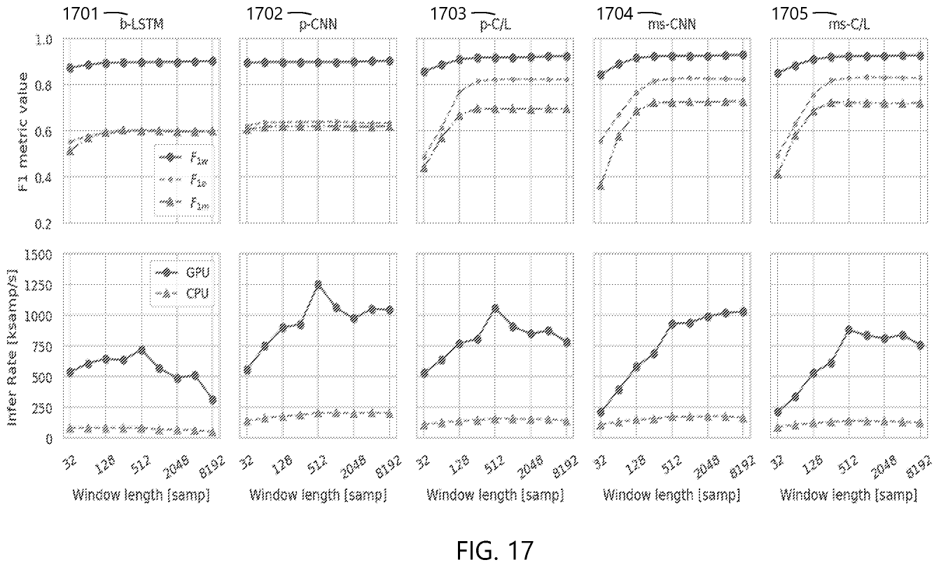

FIG. 17 illustrates the effects of changing the sliding window length used in the interference step according to certain non-limiting embodiments. The models shown in FIG. 17 are b-LSTM 1701, p-CNN 1702, p-C/L 1703, ms-CNN 1704, and ms-C/L 1705. Although one or more models can process time series of arbitrary length, in certain non-limiting embodiments efficiency and memory constraints can lead to the use of windowing. In addition, some overlap can be used to reduce edge effects in those windows. For example, a 50% overlap can be used, weighted with a Hanning window to de-emphasize edges and reduce the introduction of discontinuities where windows meet. The batch size, for example, can be 100 windows.

While model accuracy increases monotonically with window length, the inference rate can reach a maximum for LSTM-containing models where the efficiencies of constructing and reassembling longer segments, and the efficiencies of some parallel execution on the GPUs, balance the inefficient sequential execution of the LSTM layer on GPUs. While the balance can vary, windows of 256 to 2048 samples tend to perform well. On CPUs, these effects can be less prominent due to less parallelization, although some short windows can exhibit overhead. The efficiency drawbacks of executing LSTMs on GPUs can be eased by using a GPU LSTM implementation, such as the NVIDIA cuda Deep Neural Network library (cuDNN) which accelerates these computations, and by using an architecture with a large output to input stride ratio so that the input sequence to the LSTM layer can be shorter.

In certain non-limiting embodiments, one or more models do not include an LSTM layer. For example, both p-CNN and ms-CNN variants do not include an LSTM layer. Those models can have a finite ROI, and edge effects can only be possible within ROI/2 of the window ends. In other words, windows can overlap by approximately ROI/2 input samples, and the windows can simply be concatenated after discarding half of each overlapped region, without using a weighted window. When such as windowing strategy is applied, the efficiency benefit of longer windows can be even more pronounced, especially considering the excellent parallelizability of CNNs. In some examples, a batch size of 1 can be applied using the longest window length possible given system memory constraints. In some non-limiting embodiments, GPUs achieved far greater inference rates than CPUs. However, when models are small, meaning that they have few trainable parameters or are LSTM-based, CPU execution can be preferred.

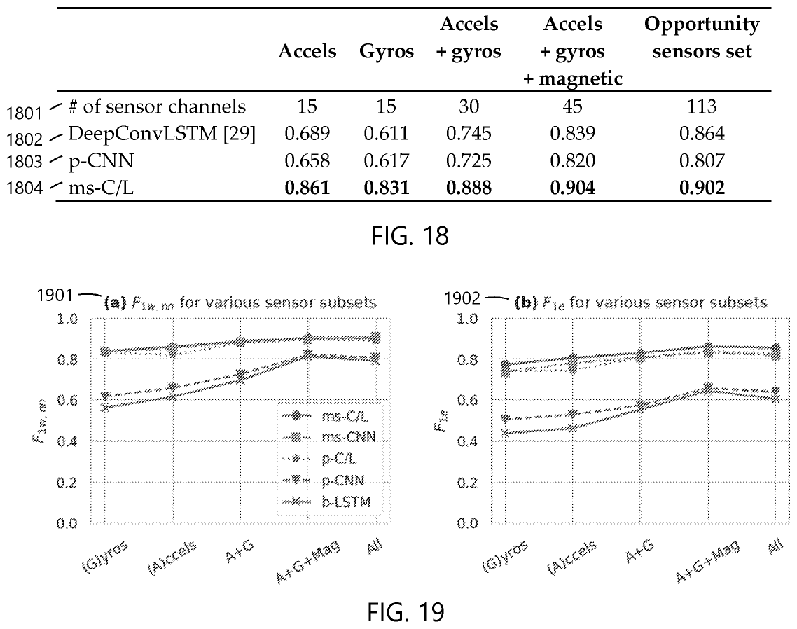

FIG. 18 illustrates performance of one or more models according to certain non-limiting embodiments based on a number of sensors. In particular, the number of different sensor channels 1801 tested can include 15 accelerometers, 15 gyroscopes, 30 accelerometers and gyroscopes, 45 accelerometers, gyroscopes, and magnetometers, as well as the 113 opportunity sensor sets. The models tested in FIG. 18 are DeepConvLSTM 1802, p-CNN 1803, and ms-C/L 1804. As shown in FIG. 18, models using accelerometers are more useful than gyroscopes, while models that use both accelerometers and gyroscopes also perform well.

In certain non-limiting embodiments the one or more models are well-suited to datasets with relatively few sensors. The models shown in FIG. 18 are trained and evaluated on the same train, validation, and test sets, but with different subsets of sensor outputs ranging from 15 to 113 channels. Model architecture parameters can be held constant, or close to constant, but the number of trainable parameters in the first model layer can vary when the number of input channels changes. Further analysis can be seen in FIG. 19, where both F_{1w,nn} and event-based F₁ₑ are plotted across the same set of sensor subsets for ms-C/L, ms-CNN, p-C/L, p-CNN, and b-LSTM.

The ms-C/L model can outperform the other models, especially according to event-based metrics. ms-C/L, ms-CNN, and p-C/L models exhibit consistent performance even with fewer sensors. The five models have long or unbounded ROIs, which can help them compensate for the missing sensor channels. In certain non-limiting embodiments, the one or more models perform best on a 45-sensor subset. This can indicate that the models can be overtrained for a sensor set larger than 45.

FIG. 19 illustrates performance analysis of models according to certain non-limiting embodiments. In particular, FIG. 19 illustrates further analysis for sample-by-sample F_{1w,nn} for various subsets 1901 and event-based F₁ₑ for various subsets 1902 plotted across the same set of sensor subsets for ms-C/L, ms-CNN, p-C/L, p-CNN, and b-LSTM. Using larger sensor subsets, including gyroscopes (G), accelerometers (A), and the magnetic (Mag) components of the inertial measurement units, as well as all 113 standard sensors channels (All), tended to improve performance metrics. Some models, such as ms-C/L, ms-CNN, and p-C/L, maintain relatively high performance even with fewer sensor channels.

The one or more models, according to some non-limiting embodiments, can be used to simultaneously calculate multiple independent outputs. For example, the same network can be used to simultaneously predict both a quickly-varying behavior and a slowly-varying posture. The loss functions for the multiple outputs can be simply added together, and the network can be trained on both simultaneously. This can allow a degree of automatic transfer learning between the two label sets.

Certain non-limiting embodiments can be used to determine multi-label classification and regression problems by changing the output types, such as changing the final activation function from softmax to sigmoid or linear, and/or the loss functions from cross-entropy to binary cross-entropy or mean squared error. In some examples the independent outputs in the same model can be combined. Further, one or more other layers can be added in certain non-limiting embodiments. Certain other embodiments can help to improve the layer modules by using skip connections or even a heterogeneous inception-like architecture. In addition, some non-limiting embodiments can be extended to real-time or streaming applications by, for example, using only CNNs or by replacing bidirectional LSTMs with unidirectional LSTMs.

While some of the data described above reflects pet activity data, in certain non-limiting embodiments other data, which does not reflect pet activity, can be processed and/or analyzed using the activity recognition time series classification algorithm to infer a desired output time series. For example, other data can include, but is not limited to, financial data, cyber security data, electronic health records, acoustic data, image or video data, human activity data, or any other data known in the art. In such embodiments, the input(s) of the time series can exist in a wide range of different domains, including finance, cyber security, electronic health record analysis, acoustic scene classification, and human activity recognition. The data, for example, can be time series data. In addition, or as an alternative, the data can be first-party, such as data obtained from a wearable device, or third-party data. Third-party data can include data that is not directly collected by a given company or entity, but rather data that is purchased from other collecting entities or companies. For example, the third-party data can be accessed or purchased using a data-management platform. First-party data, on the other hand, can include data that is directly owner and/or collected by a given company. For example, first-party data can be collected from consumers using products or services offered by the given company, such as a wearable device.

In one non-limiting embodiment, the above time series classification algorithm can be applied to motor-imagery electroencephalography (EEG) data. For example, EEG data can be collected as various subjects imagine performing one or more activities rather than physically performing the one or more activities. Using the EEG readings, the time series classification algorithm can be trained to predict the activity that the subjects are imagining. The determined classifications can be used to form a brain-computer interface that allows users to directly communicate with the outside world and/or to control instruments using the one or more imagined activities, also referred to as brain intentions.

Performance of the above example can be demonstrated on various open source EEG intention recognition datasets, such as the EEG Motor Movement/Imagery Dataset from PhysioNet. See G. Schalk et al., “BCI2000: A General-Purpose Brain-Computer Interface (BCI) System,” IEEE Transactions on Biomedical Engineering, Issue 51(6), pg. 1034-1043 (2004), available at https://www.physionet.org/content/eegmmidb/1.0.0/. In certain non-limiting embodiments, no specialized spatial or frequency-based feature extraction methods were applied to the EEG Motor Movement/Imagery Dataset. Rather, the performance can be obtained by applying the model directly to the 160 Hz EEG readings. In some examples the readings can be re-scaled to have zero mean and unit standard deviation according to the statistics of the training set. To ensure that the data is representative, data from each subject can be randomly split into training, validation, and test sets so that data from each subject is represented in one set. Trials from subjects 1, 5, 6, 9, 14, 29, 32, 39, 42, 43, 57, 63, 64, 66, 71, 75, 82, 85, 88, 90 and 102 were used as the test subjects, data from subjects 2, 8, 21, 25, 28, 40, 41, 45, 48, 49, 59, 62, 68, 69, 76, 81 and 105 were used for validation purposes, and data from the remaining 70 subjects was used as training data. Each integer can represent one test subject. Performance of the example ms-C/L model is described in Table 1 and Table 2 below:

TABLE 1
Layer detail for ms-C/L applied to the
EEG intention recognition dataset.

| | | | | | | Output |
|---|---|---|---|---|---|---|
| Component | Type | wᵢₙ | wₒᵤₜ | s | k | stride ratio¹ |
| in | input | 64 | | | | |
| A | FLM_{CNN} | 64 | 128 | 1 | 5 | 1 |
| B | FLM_{CNN} | 128 | 128 | 2 | 5 | 2 |
| B | FLM_{CNN} | 128 | 128 | 2 | 5 | 4 |
| B | FLM_{CNN} | 128 | 128 | 2 | 5 | 8 |
| B | FLM_{CNN} | 128 | 128 | 2 | 5 | 16 |
| B | FLM_{CNN} | 128 | 128 | 2 | 5 | 32 |
| B | FLM_{CNN} | 128 | 128 | 2 | 5 | 64 |
| C | FLM_{CNN} | 128 | 64 | 2 | 5 | 128 |
| C | FLM_{CNN} | 64 | 32 | 2 | 5 | 256 |
| C | FLM_{CNN} | 32 | 16 | 2 | 5 | 512 |
| D | resample | 240 | 240 | | | |
| E | FLM_{CNN} | 240 | 128 | 1 | 1 | 64 |
| F | FLM_{LSTM} | 128 | 128 | 1 | | 64 |
| G | FLM_{CNN} | 128 | 5 | 1 | 1 | 64 |
| out | output | | 5 | | | |

¹Ratio of layer output length to system input length for a given layer.

TABLE 2
Behavior F1 scores, precision, and recall for
each of the intended behaviors in the test set.

| Intended Behavior | F₁ | Precision | Recall |
|---|---|---|---|
| Eyes Closed | 0.915 | 0.887 | 0.944 |
| Left Fist | 0.801 | 0.810 | 0.793 |
| Right Fist | 0.798 | 0.799 | 0.797 |
| Both Fists | 0.636 | 0.690 | 0.591 |
| Both Feet | 0.649 | 0.680 | 0.621 |
| Mean | 0.760 | 0.773 | 0.749 |
| Weighted | 0.818 | 0.816 | 0.822 |

In certain non-limiting embodiments a system, method, or apparatus can be used to assess pet wellness. As described above, data related to the pet can be received. The data can be received from at least one of the following data sources: a wearable pet tracking or monitoring device, genetic testing procedure, pet health records, pet insurance records, and/or input from the pet owner. One or more of the above data sources can collected using separate sources. After the data is received it can be aggregated into one or more databases. The process or method can be performed by any device, hardware, software, algorithm, or cloud-based server described herein.

Based on the received data, one or more health indicators of the pet can be determined. For example, the health indicators can include a metric for licking, scratching, itching, walking, and/or sleeping by the pet. For example, a metric can be the number of minutes per day a pet spends sleeping, and/or the number or minutes per day a pet spends walking, running, or otherwise being active. Any other metric that can indicate the health of a pet can be determined. In some non-limiting embodiments, a wellness assessment of the pet can be performed based on the one or more health indicators. The wellness assessment, for example, can include evaluation and/or detection of dermatological condition(s), dermatological disease(s), ear/eye infection, arthritis, cardiac episode(s), cardiac condition(s), cardiac disease(s), allergies, dental condition(s), dental disease(s), kidney condition(s), kidney disease(s), cancer, endocrine condition(s), endocrine disease(s), deafness, depression, pancreatic episode(s), pancreatic condition(s), pancreatic disease(s), obesity, metabolic condition(s), metabolic disease(s), and/or any combination thereof. The wellness assessment can also include any other health condition, diagnosis, or physical or mental disease or disorder currently known in veterinary medicine.

Based on the wellness assessment, a recommendation can be determined and transmitted to one or more of a pet owner, a veterinarian, a researcher and/or any combination thereof. The recommendation, for example, can include one or more health recommendations for preventing the pet from developing one or more of a disease, a condition, an illness and/or any combination thereof. The recommendation, for example, can include one or more of: a food product, a pet service, a supplement, an ointment, a drug to improve the wellness or health of the pet, a pet product, and/or any combination thereof. In other words, the recommendation can be a nutritional recommendation. In some embodiments, a nutritional recommendation can include an instruction to feed a pet one or more of: a chewable, a supplement, a food and/or any combination thereof. In some embodiments, the recommendation can be a medical recommendation. For example, a medical recommendation can include an instruction to apply an ointment to a pet, to administer one or more drugs to a pet and/or to provide one ore more drugs for or to a pet.

The term “pet product” can include, for example, without limitation, any type of product, service, or equipment that is designed, manufactured, and/or intended for use by a pet. For example, the pet product can be a toy, a chewable, a food, an item of clothing, a collar, a medication, a health tracking device, a location tracking device, and/or any combination thereof. In another example a pet product can include a genetic or DNA testing service for pets.

The term “pet owner” can include any person, organization, and/or collection of persons that owns and/or is responsible for any aspect of the care of a pet.

In certain non-limiting embodiments, a pet owner can purchase a pet insurance policy from a pet healthcare provider. To obtain the insurance policy, the pet owner can pay a weekly, monthly, or yearly base cost or fee, also known as a premium. In some non-limiting embodiments, the base cost, base fee, and/or premium can be determined in relation to the wellness assessment. In other words, the health or wellness of the pet can be determined, and the base cost and/or premium that a policy holder (e.g. one or more pet owner(s)) for an insurance policy must pay can be determined based on the determined health or wellness of the pet.

In other non-limiting embodiments, a surcharge and/or discount can be determined and/or applied to a base cost or premium for a health insurance policy of the pet. This determination can be either automatic or manual. Any updates to the surcharge and/or discount can be determined periodically, discretely, and/or continuously. For example, the surcharge or discount can be determined periodically every several months or weeks. In some non-limiting embodiments, the surcharge or discount can be determined based on the data received after a recommendation has been transmitted to one or more pet owner. In other words, the data can be used to monitor and/or track whether one or more pet owners are following and/or otherwise complying with one or more provided recommendations. If a pet owner follows and/or complies with one or more of the provided recommendations, a discount can be assessed or applied to the base cost or premium of the insurance policy. On the other hand, if one or more pet owners fails to follow and/or comply with the provided recommendation(s), a surcharge and/or increase can be assessed or applied to the base cost or premium of the insurance policy. In certain non-limiting embodiments the surcharge or discount to the base cost or premium can be determined based on one or more of the data, wellness assessment, and/or recommendation.

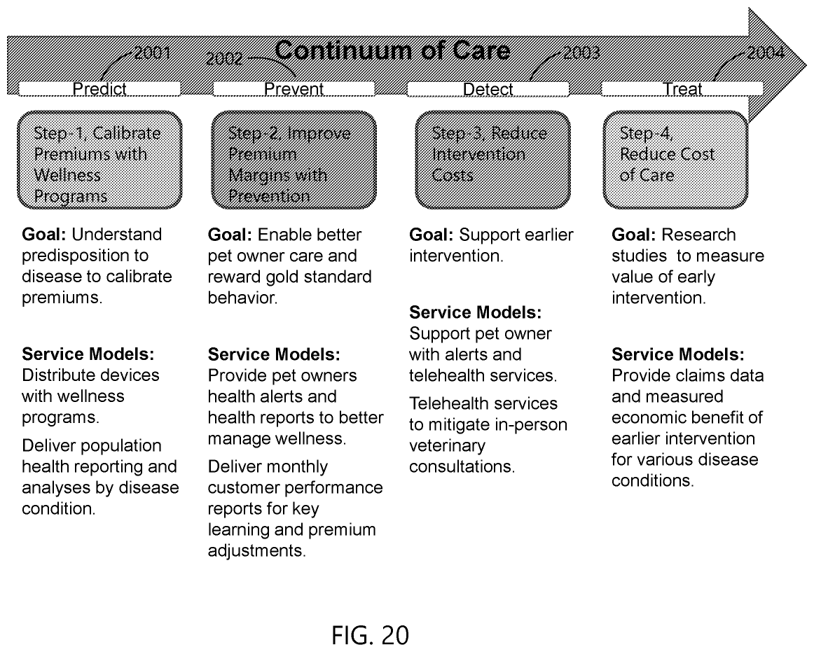

FIG. 20 illustrates a flow diagram of a process for assessing pet wellness according to certain non-limiting embodiments. In particular, FIG. 20 illustrates a continuum of care that can include prediction 2001, prevention 2002, detection 2003, and treatment 2004. In prediction step 2001, data can be used to understand or determine any health condition or predisposition to disease of a given pet. This understanding or determining of the health condition or predisposition to a disease can be a wellness assessment. It will be understood that the wellness assessment may be carried out using any method as described herein. The determined health condition or predisposition to disease can be used to determine, calculate, or calibrate base cost or premium of an insurance policy. Prediction 2001 can be used to delivery or transmit the wellness assessments and/or recommendations to a pet owner, or any other interested party. For example, in some non-limiting embodiments only the wellness assessment or recommendation can be transmitted, while in other embodiments both the wellness assessment and recommendation can be transmitted. The recommendation can also be referred to as a health recommendation, a health alert, a health card, or a health report. In certain non-limiting embodiments a wearable device, such as a tracking or monitoring device can be used to determine the recommendation.

Prevention 2002, shown in FIG. 20, includes improving premium margins on pet insurance policies. This prevention step can help to improve pet care and reward good pet owner behavior. In particular, prevention 2002 can provide pet owners with recommendations to help pet owners better manage the health of their pets. The recommendations can be provided continuously, discretely, or periodically. After the recommendations are transmitted to the pet owner data can be collected or received, which can be used to track or follow whether the pet owner is following the provided recommendation. This continued monitoring, after the transmitting of the recommendations, can be aggregated into a performance report. The performance report can then be used to determine whether to adjust the base cost or premium of a pet insurance policy.

FIG. 20 also includes detection 2003 that can be used to reduce intervention costs via early detection of potential wellness or health concerns of a pet. As indicated in prevention 2002, recommendations can be transmitted or provided to a pet owner. These recommendations can help to reduce intervention costs by detecting potential wellness or health issues early. In addition to, or as part of the recommendations, a telehealth service can be provided to pet owners. The telehealth service can replace or accompany in-person veterinary consultations. The use of telehealth services can help to reduce costs and overhead associated with in-person veterinarian consultations.

Treatment 2004 can include using the received or collected data to measure the effectiveness or value of early intervention for various disease or health conditions. In certain non-limiting embodiments, the data can detect health indicators of the pet after a recommendation is followed for a pet owner. Based on the data, health indicators, or wellness assessment, the effectiveness of the recommendation can be determined. For example, the recommendation can include administering a tropical cream or ointment to a pet to treat an assessed a skin condition. After the tropical cream or ointment is administered, data collected can help to assess the effectiveness of treating the skin condition. In certain non-limiting embodiments, metrics reflecting the effectiveness of the recommendation can be transmitted. The effectiveness of the recommendation, for example, can be clinical as related to the pet or financial as related to the pet owner.

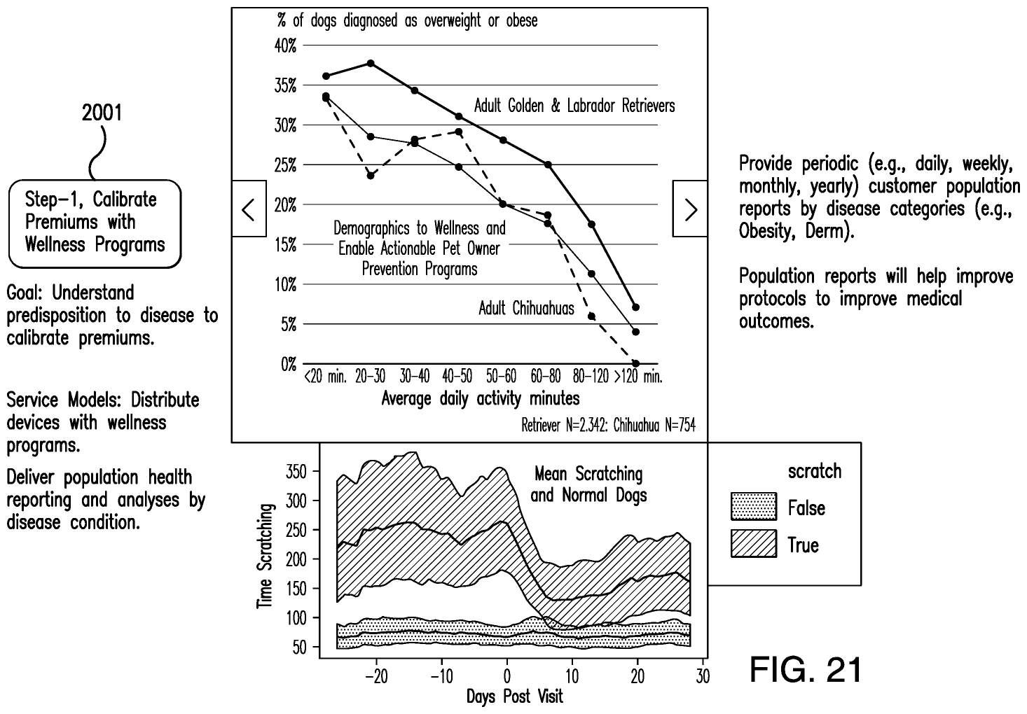

FIG. 21 illustrates an example step performed during the process for assessing pet wellness according to certain non-limiting embodiments. In particular, FIG. 21 illustrates an example of prediction 2001 shown in FIG. 20. As previously explained, prediction 2001 can use data scan be used to understand or determine any health condition or predisposition to disease of a given pet. Raw data can be used to determine health indicators, such as daily activity time or mean scratching time. Based on the data, a wellness assessment of the pet can be determined. For example, as shown in FIG. 21 37% of adult golden and labrador retrievers with an average daily activity between 20 to 30 minutes are overweight or obese. As such, if the health indicator shows a low average daily activity, the corresponding wellness assessment can be the pet being obese or overweight. The associated recommendation based on the wellness assessment can then be to increase average daily activity by at least 30 to 40 minutes.

A similar assessment can be made regarding scratching, which can be a health indicator, as shown in FIG. 21. If a given pet is scratching more than a threshold amount for a dog of a certain weight, breed, age, etc., the pet may have one or more of a dermatological condition, such as a skin condition, a dermatological disease, another dermatological issue and/or any combination thereof. An associated recommendation can then be provided to one or more of: a pet owner, a veterinarian, a researcher and/or any combination thereof. In some non-limiting embodiments, the wellness assessment can be used to determine the health of a pet and/or the predisposition of a pet to any health condition(s) and/or to any disease(s). This wellness assessment, for example, can be used to determine the base cost or premium of an insurance policy.

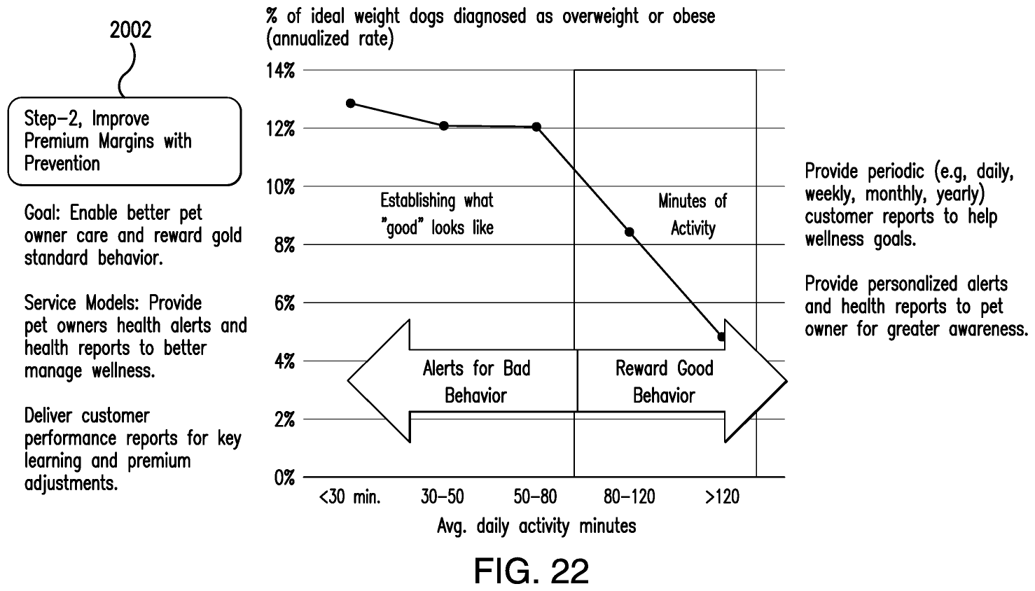

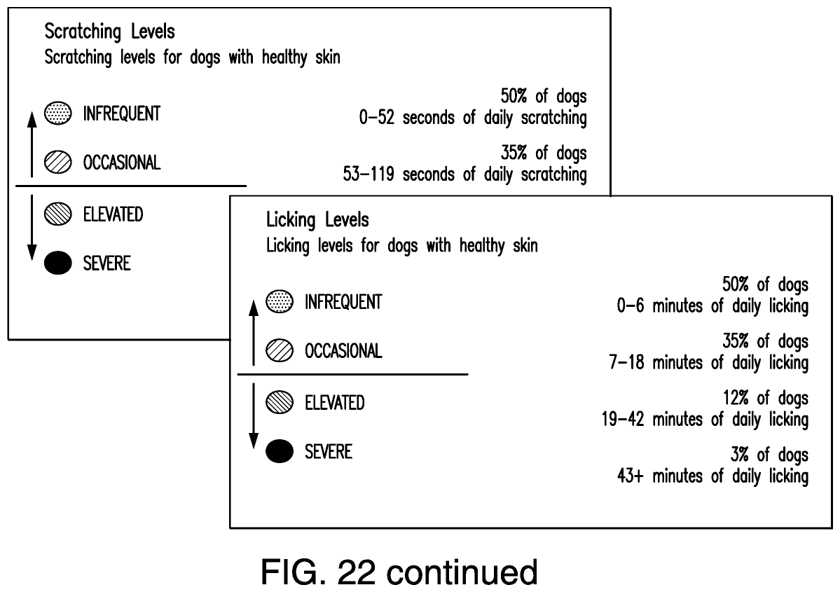

FIG. 22 illustrates an example step performed during the process for assessing pet wellness according to certain non-limiting embodiments. In particular, FIG. 22 illustrates prevention 2002 shown in FIG. 20. Based on the wellness assessment a recommendation can be determined and transmitted to the pet owner. For example, as described in FIG. 21 the recommendation can be to increase the activity time of a pet. If the pet owner follows the recommendation and increases the activity level of the average daily activity level of the pet by the recommended amount, the cost base or premium of the pet insurance policy can be lowered, decreased, or discounted. On the other hand, if the pet owner does not follow the recommendations the cost base or premium of the pet insurance policy can be increased or surcharged. In some non-limiting embodiments, additional recommendations or alerts can be sent to the user based on their compliance or non-compliance with the recommendations. The recommendations or alerts can be personalized for the pet owner based on the data collected for a given pet. The recommendations or alerts can be provided periodically to the pet owner, such as daily, weekly, monthly, or yearly. As shown in FIG. 22, other wellness assessments can include scratching or licking levels.

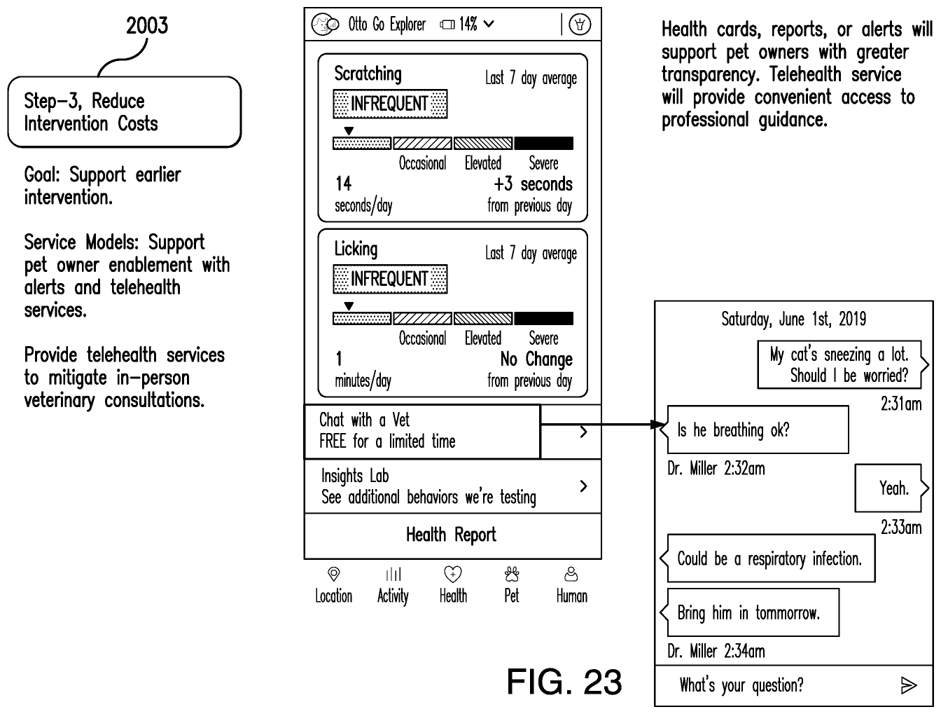

FIG. 23 illustrates an example step performed during the process for assessing pet wellness according to certain non-limiting embodiments. In particular, FIG. 23 illustrates intervention 2003 shown in FIG. 20. As shown in FIG. 23 one or more health cards, reports, or alerts can be transmitted to the pet owner to convey compliance or non-compliance with recommendations. In some non-limiting embodiments the pet owner can consult with a veterinarian or pet health care professional through a telehealth platform. Any known telehealth platform known in the art can be used to facilitate communication between the pet owner and the veterinarian pet health care professional. The telehealth visit can be included as part of the recommendations transmitted to the pet owner. The telehealth platform can be run on any user device, mobile device, or computer used by the pet owner.

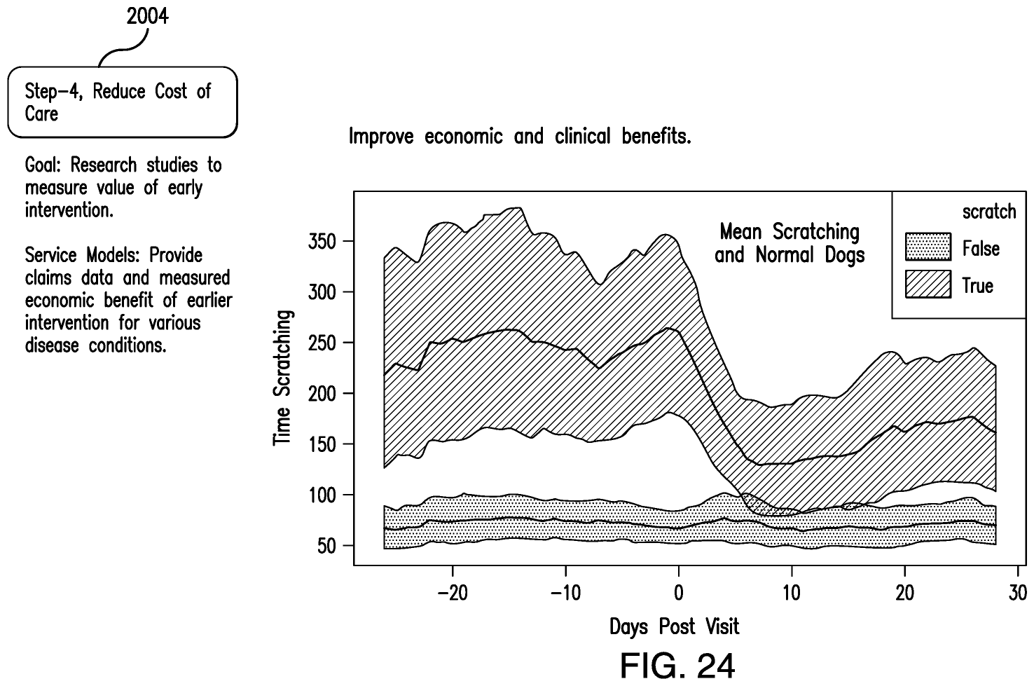

FIG. 24 illustrates an example step performed during the process for assessing pet wellness according to certain non-limiting embodiments. In particular, FIG. 24 illustrates treatment 2004 shown in FIG. 20. As shown in FIG. 24, the data collected can be used to measure the economic and health benefits of the interventions recommended in FIGS. 21 and 22. For example, a comparison of health indicators, such as scratching, after or before a pet owner follows a recommendation can help to assess the effectiveness of the recommendation.

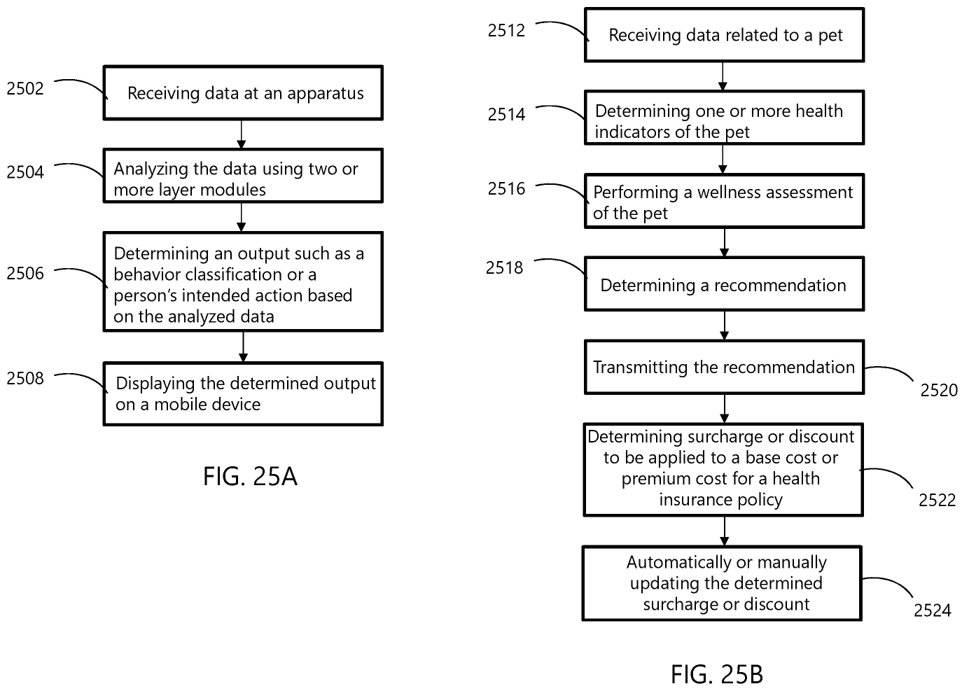

FIG. 25A illustrates a flow diagram illustrating a process for assessing pet wellness according to certain non-limiting embodiments. In particular, FIG. 25A illustrates a method or process for data analysis performed using a system or apparatus as described herein. The method or process can include receiving data at an apparatus, as shown in 2502. The data can include at least one of financial data, cyber security data, electronic health records, acoustic data, human activity data, and/or pet activity data. As shown in 2504, the method or process can include analyzing the data using two or more layer modules. Each of the layer modules can include at least one of a many-to-many approach, striding, downsampling, pooling, multi-scaling, or batch normalization. In certain non-limiting embodiments, each of the layer modules can be represented as: FLM_{type}(wₒᵤₜ, s, k, p_{drop}, b_{BN}), where the type is a convolutional neural network (CNN), wₒᵤₜ is a number of output channels, s is a stride ratio, k is a kernel length, p_{drop} is a dropout probability, and b_{BN} is a batch normalization. In some-non limiting embodiments, the two or more layers can include at least one of full-resolution convolutional neural network, a first pooling stack, a second pooling stack, a resampling step, a bottleneck layer, a recurrent stack, or an output module.

As shown in 2506, the method or process can include determining an output such as a behavior classification or a person's intended action based on the analyzed data. The output can include a wellness assessment, a health recommendation, a financial prediction, or a security recommendation. In 2508, the method or process can include displaying the determined output on a mobile device.

FIG. 25B illustrates a flow diagram illustrating a process for assessing pet wellness according to certain non-limiting embodiments. In particular, FIG. 25B illustrates a method or process performed using a system or apparatus as described herein. The method or process can include receiving data related to a pet, as shown in 2512. The data can be received from at least one of a wearable pet tracking or monitoring device, genetic testing procedure, pet health records, pet insurance records, or input from the pet owner. In some non-limiting embodiments the data can reflect pet activity or behavior. Data can be received before and after the recommendation is transmitted to the mobile device of the pet owner. Based on the data, one or more health indicators of the pet can be determined, as shown in 2514. The one or more health indicator can include a metric for licking, scratching, itching, walking, or sleeping by the pet.

A wellness assessment can be performed based on the one or more health indicators of the pet, as shown in 2516. The one or more health indicators, for example, can be a metric for licking, scratching, itching, walking, or sleeping by the pet. In 2518 a recommendation can be transmitted to a pet owner based on the wellness assessment. The wellness assessment can include comparing the one or more health indicators to one or more stored health indicators, where the stored health indicators are based on previous data related to the pet and/or to one or more other pets. The recommendation, for example, can include one or more health recommendations for preventing the pet from developing one or more of: a condition, a disease, an illness and/or any combination thereof. In other non-limiting embodiments, the recommendation can include one or more of: a food product, a supplement, an ointment, a drug and/or any combination thereof to improve the wellness or health of the pet. In some non-limiting embodiments, the recommendation can comprise one or more of: a recommendation to contact a telehealth service, a recommendation for a telehealth visit, a notice of a telehealth appointment, a notice to schedule a telehealth appointment and/or any combination thereof. The recommendation can be transmitted to one or more mobile device(s) of one or more pet owner(s), veterinarian(s) and/or researcher(s) and/or can be displayed at the mobile device of the one or more pet owner(s), veterinarian(s) and/or researcher(s), as shown in 2520. The transmitted recommendation can be transmitted to the pet owner(s), veterinarian(s) and/or researcher(s) periodically, discretely, or continuously.

In certain non-limiting embodiments, the effectiveness or efficacy of the recommendation can be determined or monitored based on the data. Metrics reflecting the effectiveness of the recommendation can be transmitted. The effectiveness of the recommendation, for example, can be clinical as related to the pet or financial as related to the pet owner.

As shown in 2522 of FIG. 25, a surcharge or discount to be applied to a base cost or premium for a health insurance policy of the pet can be determined. The discount to be applied to the base cost or premium for the health insurance policy can be determined when the pet owner follows the recommendation. The surcharge to be applied to the base cost or premium for the health insurance policy is determined when the pet owner fails to follow the recommendation. In certain non-limiting embodiments the surcharge or discount to be applied to the base cost or premium of the health insurance policy can be provided to the pet owner or provider of the health insurance policy. In some non-limiting embodiments, the base cost or premium for the health insurance policy of the pet can be determined based on the wellness assessment. The determined surcharge or discount to be applied to the base cost or premium for the health insurance policy of the pet can be automatically or manually updated after the recommendation has been transmitted to the pet owner, as shown in 2524.

In certain non-limiting embodiments, a health wellness assessment and/or recommendations can be based on data that includes information pertaining to a plurality of pets. In other words, the health indicators of a given pet can be compared to those of a plurality of other pets. Based on this comparison, a wellness assessment of the pet can be performed, and appropriate recommendations can be provided. In some non-limiting embodiments, the wellness assessment and recommendations can be customized based on the health indicators of a single pet. For example, instead of relying on data collected from a plurality of other pets, the determination can be based on algorithms or modules that are tuned or trained based wholly or in part on data or information related to the behavior of a single pet. Recommendations for pet products or services can then be customized to the behaviors or specific health indicators of a single pet.

As discussed above, the health indicators, for example, can include a metric for licking, scratching, itching, walking, or sleeping by the pet. These health indicators can be determined based on data, information, or metrics collected from a wearable device having one or more sensors or accelerometers. The collected data from the wearable device can then be processed by an activity recognition algorithm or model, also referred to as an activity recognition module or algorithm, to determine or identify a health indicator. The activity recognition algorithm or model can include two or more of the layer modules described above. After the health indicator is identified, in certain non-limiting embodiments the pet owner or caregiver can be asked to verify the correctness of the health indicator. For example, the pet owner or caregiver can receive a short message service, an alert or notification, such as a push alert, an electronic mail message on a mobile device, or any other type of message or notification. The message or notification can request the pet owner or caregiver to confirm the health indicator identified by the activity recognition algorithm or model. In some non-limiting embodiments the message or notification can indicate a time during which the data, information, or metrics were collected. If the pet owner or caregiver cannot confirm the health indicator, the pet owner or caregiver can be asked to input the activity of the pet at the indicated time.

In certain non-limiting embodiments, the pet owner or caregiver can be contacted after one or more health indicators are determined or identified. However, the pet owner or caregiver need not be contacted after each health indicator is determined or identified. Contacting the pet owner or caregiver can be an automatic process that does not require administrative intervention.

For example, the pet owner or caregiver can be contacted when the activity recognition algorithm or model has low confidence in the identified or determined health indicator, or when the identified health indicator can be unusual, such as a pet walking at night or experiencing two straight hours of scratching. In some other non-limiting embodiments, the pet owner or caregiver need not be contacted when the pet owner or caregiver is not around their pet during the indicated time in which the health indicator was identified or determined. To determine that the pet owner or caregiver is not around their pet, the reported location from the pet owner or caregiver's mobile device can be compared to the location of the wearable device. Such a determination can utilize short-distance communication methods, such as Bluetooth, or any other known method to determine proximity of the mobile device to the wearable device.

In some non-limiting embodiments, the pet owner or caregiver can be contacted after one or more predetermined health indicators are identified. The predetermined health indicators, for example, can be chosen based on lack of training data or health indicators for which the activity recognition algorithm or model experiences low precision or recall. The pet owner can then input a response, such as a confirmation or a denial of the health indicator or activity, using, for example, a GUI on a mobile device. The pet owner or caregiver's response can be referred to as feedback. The GUI can list one or more pet activities or health indicators. The GUI can also include an option for a pet owner or caregiver to select that is neither a denial nor a confirmation of the health indicator or pet activity.

When the pet owner or caregiver confirms the one or more health indicators during the indicated time, the activity training model can be further trained or tuned based on the pet owner or caregiver's confirmation. For example, the inputted data used to train or tune the activity recognition algorithm or model can be accelerometer or sensor data a given period of time before or after the indicated time. The output of the activity recognition algorithm or model can be a high probability of about 1 of the pet licking across the indicated time period. The method, process, or system can keep track of which activities the pet owner or caregiver did not confirm so that they can be ignored during the model training process.

In certain non-limiting embodiments, the pet owner or caregiver can deny the occurrence of the one or more health indicators during the indicated time and does not provide information related to the pet's activity during the indicated time. The pet owner can be an owner of the pet, while a caregiver can be any other person who is caring for the pet, such as a pet walker, veterinarian, or any other person watching the pet. In such embodiments, the inputted data used to train or tune the activity recognition algorithm or model can be accelerometer or sensor data a given period of time before or after the indicated time. The output of the activity recognition algorithm or model can be a low probability of about 0 of the pet activity. The method, process, or system can keep track of which activities the pet owner or caregiver denied so that they can be ignored during the model training process.

In other non-limiting embodiments, the pet owner or caregiver can deny the occurrence of the one or more health indicators during the indicated time, and provide information related to the pet's activity during the indicated time. In such embodiments, the inputted data used to train or tune the activity recognition algorithm or model can be accelerometer or sensor data a given period of time before or after the indicated time. The output of the activity recognition algorithm or model can be a low probability of about 0 of the identified health indicator, and a high probability of about 1 of the pet activity or health indicator inputted by the pet owner or caregiver. The method, process, or system can keep track of which activities the pet owner or caregiver denied so that they can be ignored during the model training process.

In some non-limiting embodiments, the pet owner or caregiver does not deny or confirm the occurrence. In such embodiments, the pet owner or caregiver's response or input can be excluded from the training set.

The input or response provided by the pet owner or caregiver can be inputted into the training dataset of the activity recognition model or algorithm. The activity recognition module can be a deep neutral network (DNN) trained using well known DNN training techniques, such as stochastic gradient descent (SGD) or adaptive moment estimation (ADAM). In other embodiments, the activity recognition module can include one or more layer modules described herein. During training or tuning of the activity recognition model, the health indicators not indicated by the pet owner or caregiver can be removed from the calculation of the model, with the associated classification loss weighted appropriately to help train the deep neural network. In other words, the deep neutral network can be trained or tuned based on the input of the pet owner or caregiver. By training or tuning using the input of the pet owner or caregiver, the deep neural network can help to better recognize the health indicators, thereby improving the accuracy of the wellness assessment and associated recommendations. The training or tuning of the deep neural network based on the pet owner or caregiver's response can be based on sparse training, which allows the deep neutral network to account for low-quality or partial data.

In certain non-limiting embodiments, the response provided by the pet owner or caregiver go beyond a simple correlation with the sensor or accelerometer data of the wearable device. Instead, the response can be used to collect and annotate additional data that can be used to train the activity recognition model and improve the wellness assessment and/or provided recommendations. The incorporation of the pet owner or caregiver's responses into the training dataset can be automated. Such embodiments can be more efficient and less cost intensive than having to confirm the determined or identified health indicators via a video. In some non-limiting embodiments, the automated process can identify prediction failures of the activity recognition model, add the identified failures to the training database, and/or re-train or re-deploy the activity recognition model. Prediction failures can be determined based on the response provided by the pet owner or caregiver.

In some non-limiting embodiments, a recommendation can be provided to the pet owner or caregiver based on the performed wellness assessment. The recommendation can include a pet product or service. In certain non-limiting embodiments, the pet product or service can automatically be sent to a pet owner or caregiver based on the determined recommendation. The pet owner or caregiver can subscribe or opt-in to this automatic purchase and/or transmittal of recommended pet product or services. For example, the determine health indicator can be that a pet is excessively scratching based on the data collected from a wearable device. Based on this health indicator a wellness assessment can be performed finding that the pet is experiencing a dermatological issue. To deal with this dermatological issue a recommendation for a skin and coat diet or a flea/tick relief product. The pet products associated with the recommendation can then be transmitted automatically to the pet owner or caregiver, without the pet owner or caregiver having to input any information or approve the purchase or recommendation. In other non-limiting embodiments, the pet owner or caregiver can be asked to affirmatively approve a recommendation using an input. In addition to alerting the pet owner or caregiver, the wellness assessment and/or recommendation can be transmitted to a veterinarian. The transmittal to the veterinarian can also include a recommendation to schedule for a visit with the pet, as well as a recommended consultation via a telemedicine service. In yet another embodiment, any other pet related content, instructions, and/or guidance can be transmitted to the pet owner, caregiver, or pet care provider, such as a veterinarian.

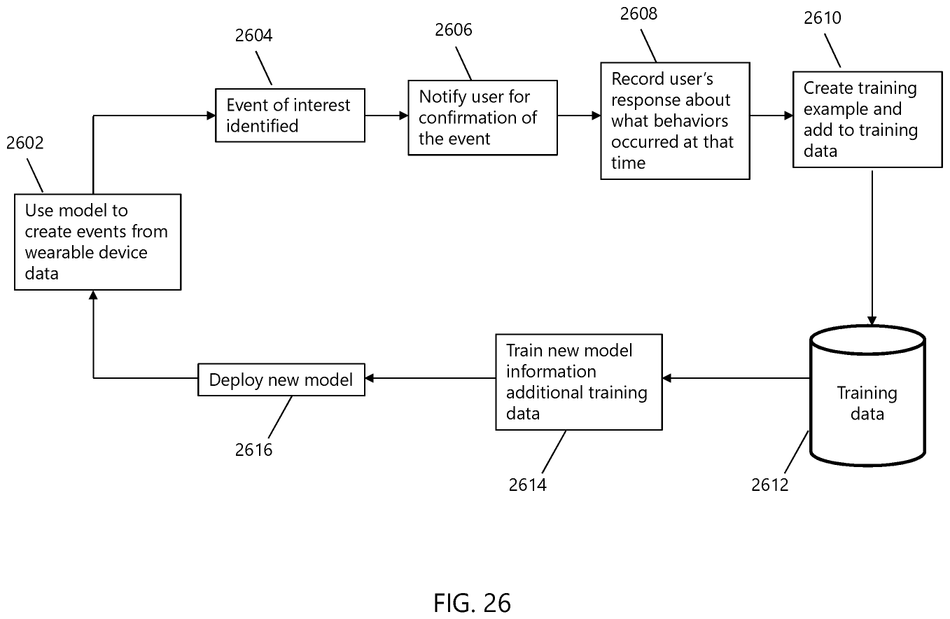

FIG. 26 illustrates a flow diagram illustrating a process for assessing pet wellness according to certain non-limiting embodiments. In particular, FIG. 26 illustrates a method or process for data analysis performed using a system or apparatus as described herein. In 2602, an activity recognition model can be used to create events from wearable device data. In other words, the activity recognition model can be used to determine or identify a health indicator based on data collected from the wearable device. In 2604, the event of interest, also referred to as a health indicator, can be identified. The pet owner or caregiver can then be asked to confirm or deny whether the health indicator, which indicated a pet's behavior or activity, occurred at an indicated time, as shown in 2608. Based on the pet owner or caregiver's response, a training example can be created and added to an updated training dataset, as shown in 2610 and 2612. The activity recognition model can then be trained or tuned based on the updated training dataset, as shown in 2614. The trained or tuned activity recognition model can then be used to recognize one or more health indicators, perform a wellness assessment, and determine a health recommendation, as shown in 2616. The trained or tuned activity recognition model can be said to be customized or individualized to a given pet.

As shown in FIG. 26, the determining of the one or more health indicators can include processing the data via an activity recognition model. The one or more health indicators can be based on an output of the activity recognition model. The activity recognition model, for example, can be a deep neural network. In addition, the method or process can include transmitting a request to a pet owner or caregiver to provide feedback on the one or more health indicators of the pet. The feedback can then be received from the pet owner or caregiver, and the activity recognition model can be trained or tuned based on the feedback from the pet owner or caregiver. In addition, or as an alternative, the activity recognition model can be trained or tuned based data from one or more pets.

In certain non-limiting embodiments, the effectiveness of the recommendation can be determined. For example, after a recommendation is transmitted or displayed, a pet owner or caregiver can enter or provide feedback indicating which of the one or more recommendations the pet owner or caregiver has followed. In such embodiments, a pet owner or caregiver can indicate which recommendation they have implemented, and/or the date and/or time when they began using the recommended product or service. For example, a pet owner or caregiver can begin feeding their pet a recommended pet food product to deal with a diagnosed or determined dermatological problem. The pet owner or caregiver can then indicate that they are using the recommended pet food product, and/or that they started using the product a certain number of days or weeks after the recommendation was transmitted or displayed on their mobile device. This feedback from the pet owner or caregiver can be used to track and/or determine the effectiveness of the recommendation. The effectiveness can then be reported to the pet owner or caregiver, and/or further recommendations can be made based on the determined effectiveness. For example, if the indicated pet food product has not improved a tracked health indicator, a different pet product or service can be recommended. On the other hand, if the indicated pet food product has improved the tracked health indicator, the pet owner or caregiver can receive an indication that the recommended pet food product has improved the health of the pet.

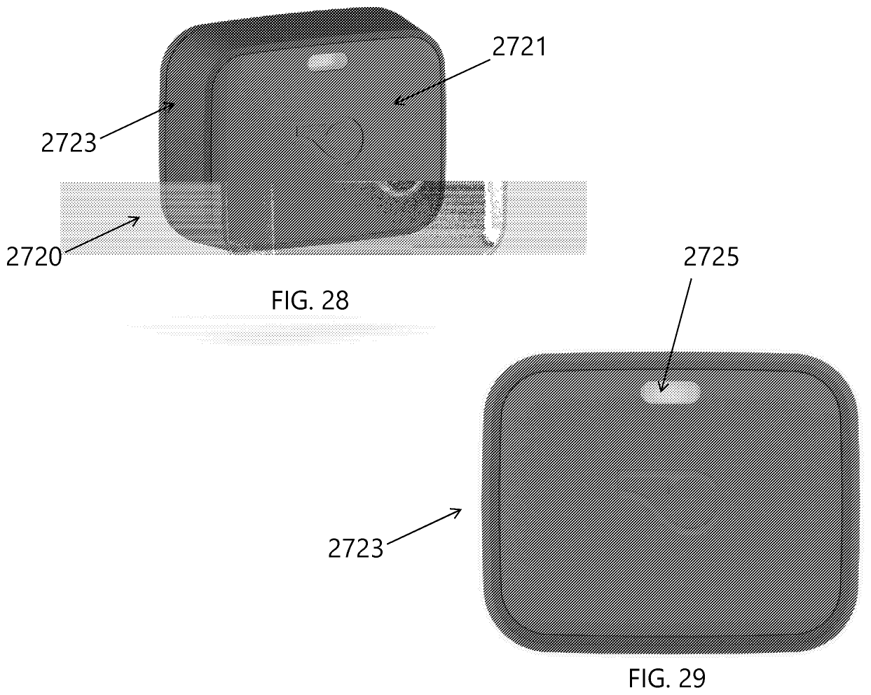

As noted above, the tracking device according to the disclosed subject matter can comprise a computing device designed to be worn, or otherwise carried, by a user or other entity, such as an animal. The wearable device can take on any shape, form, color, or size. In one non-limiting embodiment, the wearable device can be placed on or inside the pet in the form of a microchip. Additionally or alternatively, and as embodied herein, the tracking device can be a wearable device that is couplable with a collar band. For example, the wearable device can be attached to a pet collar. FIG. 27 is a perspective view of a collar 2700 having a band 2710 with a tracking device 2720, according to an embodiment the disclosed subject matter. Band 2710 includes buckle 2740 and clip 2730. FIG. 28 shows a perspective view of the tracking device 2720 and FIG. 29 shows a front view of the device 2720.

As shown in FIG. 29, the wearable device 2720 can be rectangular shaped. In other embodiments the wearable device 2720 can have any other suitable shape, such as oval, square, or bone shape. The wearable device 2720 can have any suitable dimensions. For example, the device dimensions can be selected such that a pet can reasonably carry the device. For example, the wearable device can weigh 0.91 ounces, have a width of 1.4 inches, a height or length of 1.8 inches, and a thickness or depth of 0.6 inches. In some non-limiting embodiments wearable device 2720 can be shock resistant and/or waterproof.

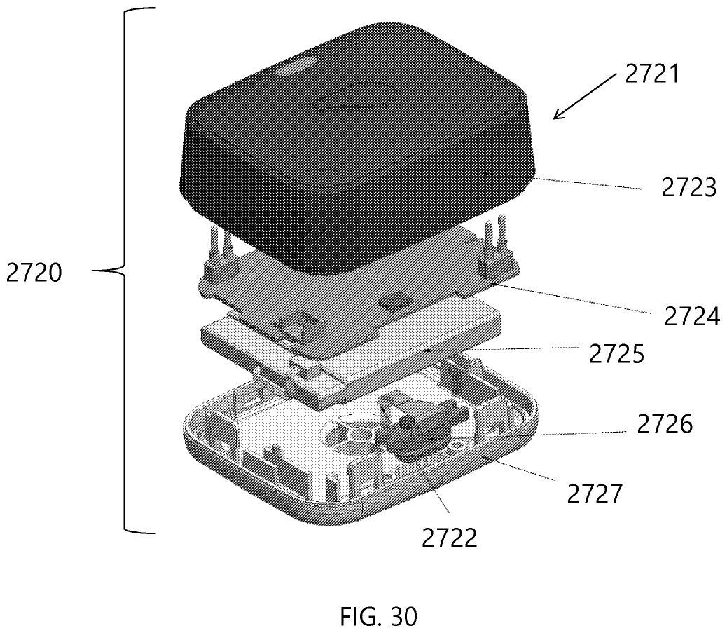

The wearable device comprises a housing that can include a top cover 2721 and a base 2727 coupled with the top cover. Top cover 2721 includes one or more sides 2723. As shown in the exploded view of FIG. 30, the housing can further include the inner mechanisms for the functional operation of the wearable device, such as a circuit board 2724 having a data tracking assembly and/or one or more sensors, a power source such as a battery 2725, a connector such as a USB connector 2726, and inner hardware 2727, such as a screw, to couple the device together, amongst other mechanisms.

The housing can further include an indicator such as an illumination device (such as but not limited to a light or light emitting diode), a sound device, and a vibrating device. The indicator can be housed within the housing or can be positioned on the top cover of the device. As best shown in FIG. 29, an illumination device 2725 is depicted and embodied as a light on the top cover. However, the illumination device can alternatively be positioned within the housing to illuminate at least the top cover of the wearable device. In other embodiments, a sound device and/or a vibrating device can be provided with the tracking device. The sound device can include a speaker and make sounds such as a whistle or speech upon a trigger event. As discussed herein, the indicator can be triggered upon a predetermined geo-fence zone or boundary.

In certain non-limiting embodiments, the illumination device 2725 can have different colors indicating the charge level of the battery and/or the type of radio access technology to which wearable device 2720 is connected. In certain non-limiting embodiments, illumination device 2725 can be the illumination device described in FIG. 4. In other words, the illumination device 2725 can be activated manually or automatically once the pet exits the geo-fence zone. Alternatively, or in addition to, a user can manually activate illumination device 2725 using an application on the mobile device based on data received from the wearable device. Although illumination device 2725 is shown as a light, in other embodiments not shown in FIG. 29, the illumination device can be replaced with an illumination device, a sound device, and/or a vibrating device.

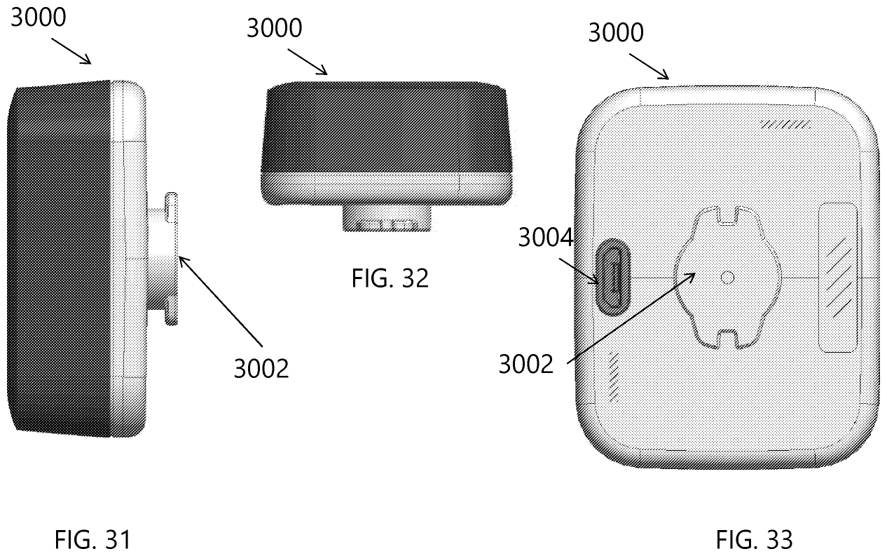

FIGS. 31-33 show the side, top, and bottom views of the wearable tracking device 3000, which can be similar to wearable device 2720 shown in FIGS. 27-30. As depicted, the housing can further include an attachment device 3002. The attachment device 3002 can couple with a complementary receiving plate and/or to the collar band 3002, as further discussed below with respect to FIG. 42. The housing can further include a receiving port 3004 to receive a cable, as further discussed below with respect to FIG. 33.

As shown in FIGS. 27-30, the top cover 2721 of wearable device 2720 includes a top surface and one or more sidewalls 2723 depending from an outer periphery of the top surface, as best shown in FIGS. 28 and 30. In one non-limiting embodiment, the top cover is separable from the sidewall and can further be separately constructed units that are coupled together. In alternative embodiments, the top cover is monolithic with the sidewall. The top cover can comprise a first material and the sidewall can comprise a second material such that the first material is different from the second material. In other embodiments, the first and second material are the same. In the embodiments of FIGS. 28 and 30, the top surface of top cover 2721 is a different material than the one or more sidewalls 2723.

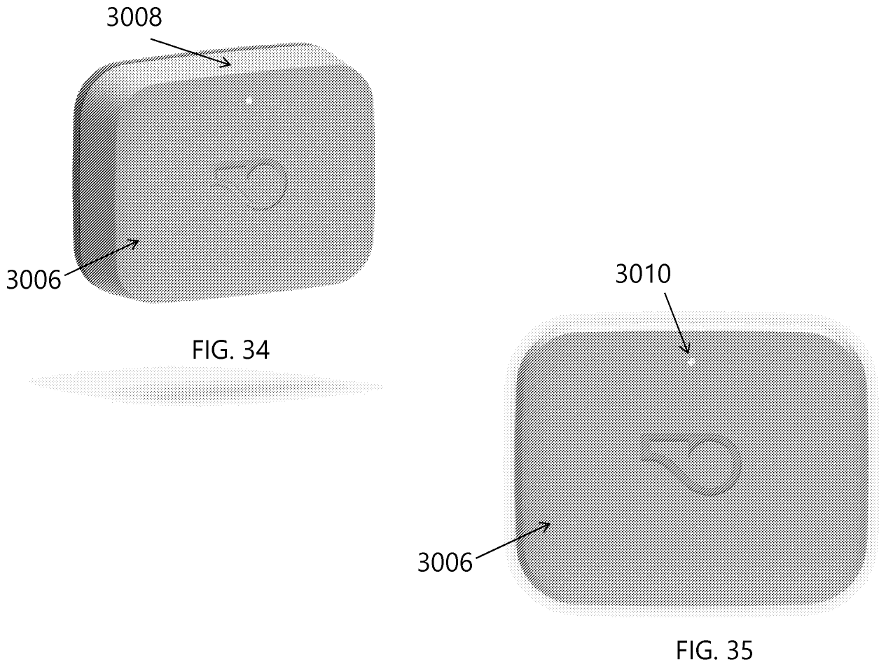

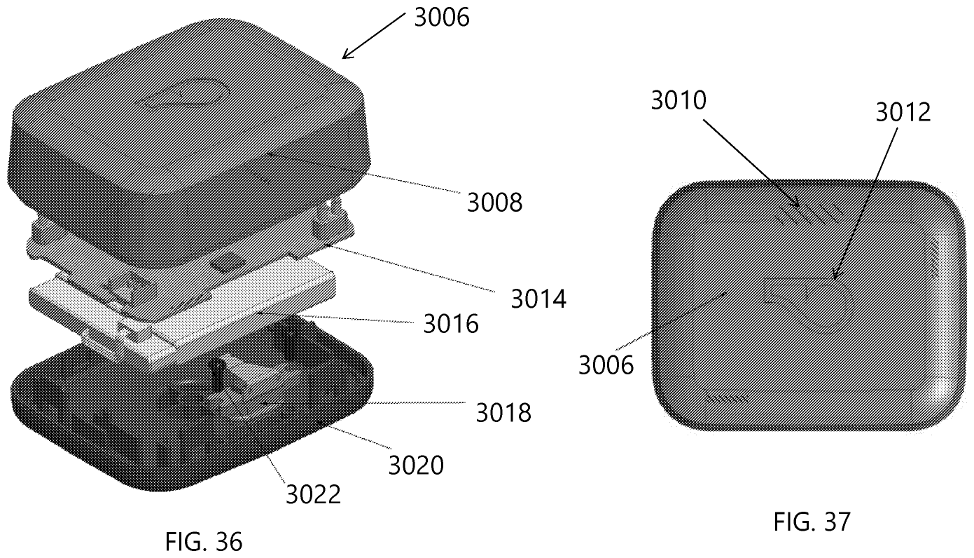

FIG. 34 depicts a perspective view of a tracking device according to another embodiment of the disclosed subject matter. In this embodiment, top surface 3006 of the top cover is monolithic with one or more sidewalls 3008 and is constructed of the same material. FIG. 35 shows a front view of the tracking device of FIG. 34. In this embodiment, the top cover includes an indicator embodied as a status identifier 3010. The status identifier can communicate a status of the device, such as a charging mode (reflective of a first color), an engagement mode (such as when interacting with a Bluetooth communication and reflective of a second color), and a fully charged mode (such as when a battery life is above a predetermined threshold and reflective of a third color). For example, when the status identifier 3010 is amber colored the wearable can be charging. On the other hand, when status identifier 3010 is green the battery of the wearable device can be said to be fully charged. In another example, status identifier 3010 can be blue, meaning that wearable device 3000 is either connected via Bluetooth and/or currently communicating with another device via a Bluetooth network. In certain non-limiting embodiments, the wearable device using the Bluetooth Low Energy (BLE) can be advantageous. BLE can be a wireless personal network that can help to reduce power and resource consumption by the wearable device. Using BLE can therefore help to extend the battery life of the wearable device. Other status modes and colors thereof of status identifier 3010 are contemplated herein. The status identifier can furthermore blink or have a select pattern of blinking that can be indicative of a certain status. The top cover can include any suitable color and pattern, and can further include a reflective material or a material that glows in the dark. FIG. 36 is an exploded view of the embodiment of FIG. 34, but having a top cover in a different color for purposes of example. Similar to FIG. 30, the wearable device shown in FIG. 36 includes circuit 3014, battery 3016, charging port 3018, mechanical attachment 3022, and/or bottom cover 3020.

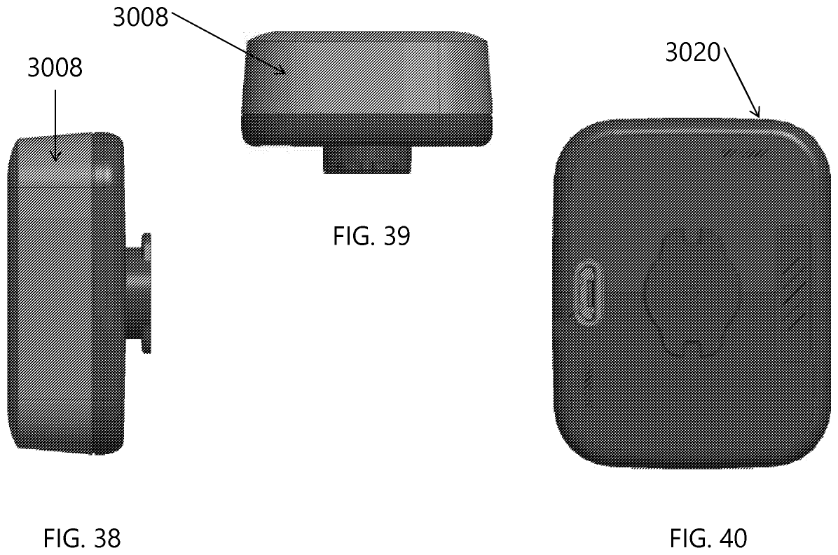

The housing, such as the top surface, can include indicia 3012, such as any suitable symbols, text, trademarks, insignias, and the like. As shown in the front view of FIG. 37, a whistle insignia 3012 is shown on the top surface of the device. Further, the housing can include personalized features, such as an engraving that features the wearer's name or other identifying information, such as a pet owner name and phone number. FIGS. 38-40 show the side, top, and bottom views of the tracking device 3000, which can further include the above noted indicia, as desired.

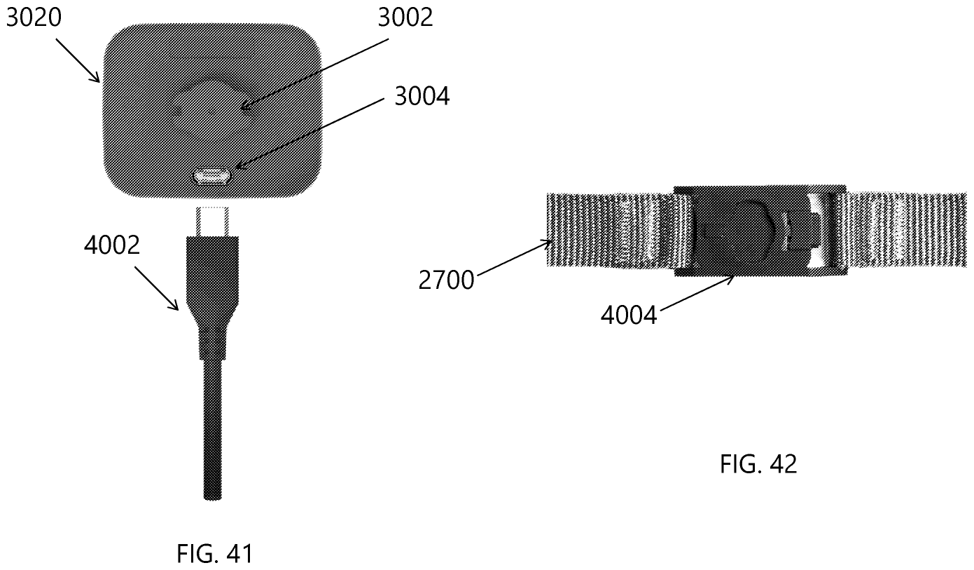

FIG. 41 depicts a back view of the tracking device couplable with a cable 4002, according to the disclosed subject matter. The cable 4002, such as a USB cable or the like, can be inserted within the port 3004 to transmit data and/or to charge the device.

As shown in FIG. 27, the tracking device can couple with the collar band 2700. The device can couple with the band in any suitable manner as known in the art. In one non-limiting embodiment, the housing, such as via the attachment device on the base, can couple with a complementary receiving plate 4004 and/or directly to the collar band 2700. FIG. 42 depicts an embodiment in which the band includes the receiving plate 4004 that will couple with the tracking device.

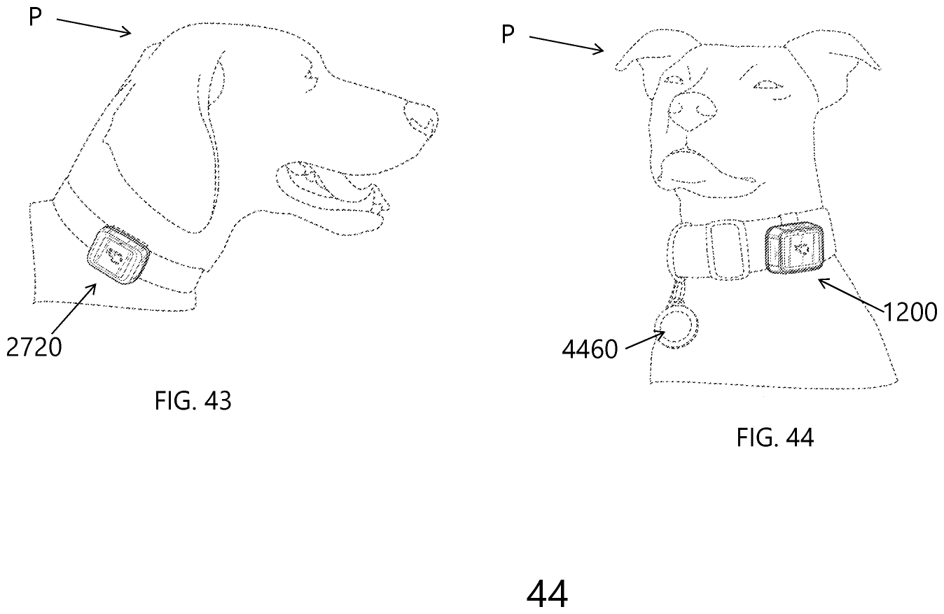

As shown in FIG. 27, the band 2700 can further include additional accessories as known in the art. In particular, the band 2700 can include adjustment mechanisms 2730 to tighten or loosen the band and can further include a clasp to couple the band to a user, such as a pet. Any suitable clasping structure and adjustment structure is contemplated here. FIGS. 43 and 44 depict embodiments of the disclosed tracking device coupled to a pet P, such as a dog, via the collar band. As shown in FIG. 44, the band can further include additional accessories, such as a name plate 4460.

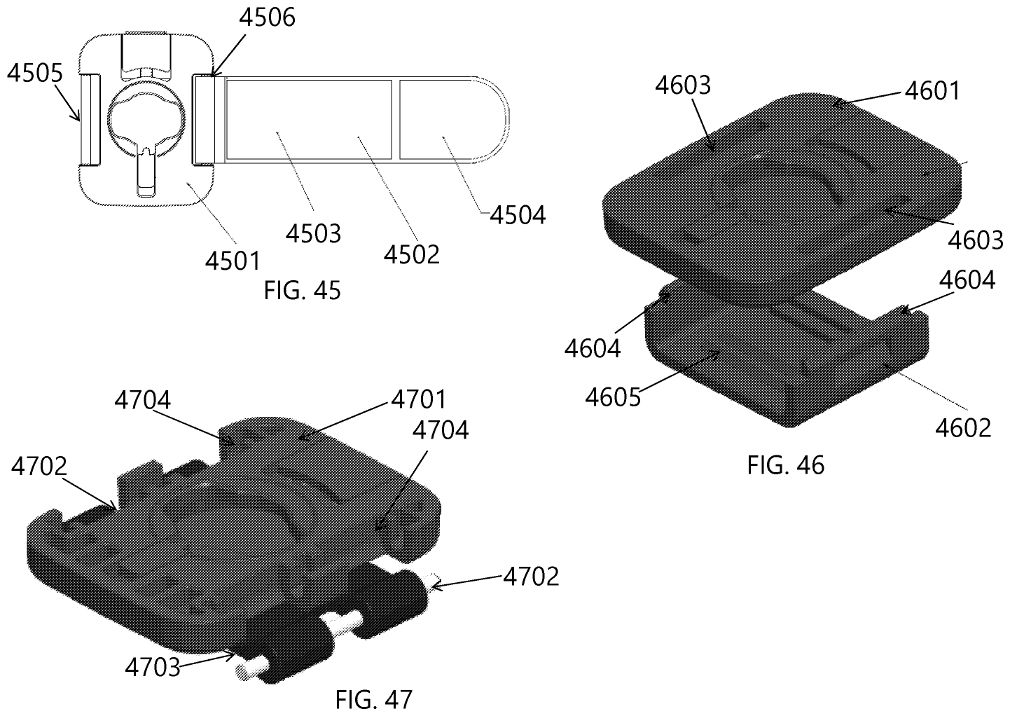

As described above, FIG. 45 depicts a receiving plate and/or support frame and collar band 2700. The support frame can be used to couple a tracking device to the collar band 2700. Attachment devices for use with tracking devices in accordance with the disclosed subject matter are described in U.S. Provisional Patent Application No. 62/768,414, titled “Collar with Integrated Device Attachment,” filed on Nov. 16, 2018, the content of which is hereby incorporated in its entirety. As embodied herein, the support frame can include a receiving aperture and latch for coupling with the attachment device and/or insertion member of the tracking device.

The collar band 2700 can couple with the support frame. For purpose of example, and as embodied in FIG. 42, the collar band can include loops for coupling with the support frame. Additionally, or alternatively, it can be desirable to couple tracking devices in accordance with the disclosed subject matter to collars without loops or other suitable configuration for securing a support frame. With reference to FIGS. 45-47, support frames can be configured with collar attachment features to secure the support frame to an existing collar.

For example, and with reference to FIG. 45, the support frame can include a hook and loop collar attachment feature. A strap 4502 can be attached to bar 4506 on the support frame. The strap 4502 can include a hook portion 4504 having a plurality of hooks, and a loop portion 4503 having a plurality of loops. The support frame 4501 can be fastened to a collar (not depicted) by passing the strap 4502 around the collar, then passing the strap around bar 4505 on the support frame 4501. After the strap 4502 has been passed around the collar and bar 4505, the hook portion 4504 can be engaged with the hook portion 4503 to secure the support frame 4501 to the collar. While reference has been made herein to using strap 4502 to secure the support frame 4501 to a collar, it is to be understood that the strap 4502 can also serve the functionality of a collar. The length of the strap 4502 can be adjusted based on the desired configuration of the attachment feature.

With reference to FIG. 46, support frame 4601 can be secured to a collar using snap member 4602. As embodied herein, the support frame 4601 can include grooves 4603 configured to receive tabs 4604 on snap member 4602. The support frame can be fastened to a collar (not depicted) by passing the collar through channel 4605 in the snap member 4602 and engaging the tabs of the snap member with the grooves 4603 of the support frame 4601. The tabs 4604 can include a lip or ridge to prevent separation of the snap member 4612 from the support frame 4601.

Additionally, or alternatively, and with reference to FIG. 47, support frame 4701 can be secured to a collar using a strap 4703 with bars 4702. As embodied herein, the support frame 4701 can include channels 4704 on opposing sides of the support frame. The channels 4704 can be configured to receive and retain bars 4702 therein. Bars 4702 can be attached to a strap 4703. For purpose of example, strap 4703 can be made of a flexible material such as rubber. The support frame 4701 can be fastened to a collar (not depicted) by passing the strap 4703 around the collar and securing bars 4702 within channels 4704 in the support frame 4701.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module can be stored on a computer readable medium for execution by a processor. Modules can be integral to one or more servers, or be loaded and executed by one or more servers. One or more modules can be grouped into an engine or an application.

For the purposes of this disclosure the term “user”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data.

Those skilled in the art will recognize that the methods and systems of the present disclosure can be implemented in many manners and as such are not to be limited by the foregoing exemplary embodiments and examples. In other words, functional elements being performed by single or multiple components, in various combinations of hardware and software or firmware, and individual functions, can be distributed among software applications at either the client level or server level or both. In this regard, any number of the features of the different embodiments described herein can be combined into single or multiple embodiments, and alternate embodiments having fewer than, or more than, all of the features described herein are possible.

Functionality can also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that can be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications can be made to the elements and operations described above to obtain a result that remains within the scope of the systems and processes described in this disclosure.

While the disclosed subject matter is described herein in terms of certain preferred embodiments, those skilled in the art will recognize that various modifications and improvements can be made to the disclosed subject matter without departing from the scope thereof. Additional features known in the art likewise can be incorporated, such as disclosed in U.S. Pat. No. 10,142,773, U.S. Publication No. 2014/0290013, U.S. Design application Nos. 29/670,543, 29/580,756, and U.S. Provisional Application Nos. 62/768,414, 62/867,226, and 62/970,575, which are each incorporated herein in their entireties by reference herein. Moreover, although individual features of one non-limiting embodiment of the disclosed subject matter can be discussed herein or shown in the drawings of the one non-limiting embodiment and not in other embodiments, it should be apparent that individual features of one non-limiting embodiment can be combined with one or more features of another embodiment or features from a plurality of embodiments.

1. A computer-implemented method for monitoring pet activity, the method comprising:
receiving data related to a pet from a wearable device comprising a sensor;
determining based on the data one or more health indicators of the pet;
performing a wellness assessment of the pet based on the one or more health indicators of the pet;
transmitting the wellness assessment of the pet to a mobile device; and
displaying the wellness assessment of the pet at the mobile device using a graphical user interface,
wherein the determining further comprises:
processing the data via an activity recognition model; and
determining the one or more health indicators based on an output of the activity recognition model.

2-4. (canceled)

5. The method according to claim 1, wherein the activity recognition model is a deep neural network comprising two or more layer modules, wherein each of the layer modules includes at least one of a many-to-many approach, striding, downsampling, pooling, multi-scaling, or batch normalization.

6. (canceled)

7. The method according to claim 1, wherein each of the layer modules can be represented as: FLM_{type}(wₒᵤₜ, s, k, p_{drop}, b_{BN}), where the type is a convolutional neural network (CNN), wₒᵤₜ is a number of output channels, s is a stride ratio, k is a kernel length, p_{drop} is a dropout probability, and b_{BN} is a batch normalization.

8. The method according to claim 1, further comprising:
transmitting a request to a pet owner or caregiver to provide feedback on the one or more health indicators of the pet;
the feedback from the pet owner or caregiver; and
training the activity recognition model based on the feedback from the pet owner or caregiver.

9.-11. (canceled)

12. The method according to claim 1, wherein the one or more health indicators comprise a metric for licking, scratching, itching, walking, or sleeping by the pet.

13. The method according to claim 1, wherein the data comprises a location of the pet, wherein the location is determined using a global positioning system.

14-16. (canceled)

17. The method according to claim 1, further comprising:
determining a health recommendation or fitness nudge for the pet based on the wellness assessment; and
transmitting the health recommendation or fitness nudge to the mobile device.

18.-20. (canceled)

21. A wearable device, comprising:
a housing, wherein the housing comprises
a top cover,
a base coupled with the top cover,
a sensor for monitoring data related to a pet,
a transceiver for transmitting the data related to the pet,
an indicator, and
wherein the indicator is at least one of an illumination device, a sound device, or a vibrating device and the indicator is positioned on the top cover.

22. The wearable device according to claim 21, wherein the indicator is configured to be turned on after the wearable device has exited a geo-fence zone.

23.-26. (canceled)

27. The wearable device according to claim 21, wherein the illumination device is positioned within the housing and includes a light or a light emitting diode configured to illuminate at least the top cover of the wearable device.

28. (canceled)

29. The wearable device according to claim 21, wherein the top cover includes a top surface and a sidewall depending from an outer periphery of the top surface.

30. (canceled)

31. The wearable device according to claim 21, wherein the housing defines a receiving port to receive a cable therein.

32. The wearable device according to claim 21, wherein the housing includes an attachment device, wherein the attachment device is coupled to a collar band.

33. A method for monitoring pet activity, the method comprising:
monitoring a location of a wearable device;
determining that the wearable device has exited a geo-fence zone based on the location of the wearable device; and
instructing the wearable device to turn on an indicator after determining that the wearable device has exited the geo-fence zone, wherein the indicator is at least one of an illumination device, a sound device, or a vibrating device.

34. The method according to claim 33, further comprising:
determining that the wearable device has entered the geo-fence zone; and
turning off the indicator when the wearable device has entered the geo-fence zone.

35. The method according to claim 34, wherein the determining that the wearable device has entered the geo-fence zone is a determination that the wearable device has re-entered the geo-fence zone after having exited the geo-fence zone.

36. The method according to claim 33,
further comprising:
receiving instructions from a mobile device to turn off the indicator, wherein the mobile device comprises an application that allows a user to turn off the indicator.

37. The method according to claim 33, wherein the instructing of the wearable device to turn on the indicator comprises turning on a light or a light emitting diode of the illumination device.

38. The method according to claim 33, further comprising:
receiving the location of the wearable device via a global positioning system (GPS) receiver.

39. The method according to claim 33, wherein the monitoring of the location of the wearable device further comprises:
identifying an active wireless network within a vicinity of the wearable device; or
identifying that the active wireless network is no longer in the vicinity of the wearable device.

40.-74. (canceled)
